SEQUENCE LISTING (4 sequences)

SEQ ID NO: 1 - protein, 170 aa, Homo sapiens
KDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYK

SEQ ID NO: 2 - protein, 170 aa, synthetic construct
  source 1..170: note = CH1mu
KDYFPEPVTVAWCSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYK

SEQ ID NO: 3 - protein, 170 aa, synthetic construct
  source 1..170: note = CH2mu
KDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYK

SEQ ID NO: 4 - protein, 9 aa, synthetic construct
  source 1..9: note = atypical glycosylation motif
TVSWNSGAL

(19) United States
(12) Patent Application Publication
(10) Pub. No.: US 2024/0409616 A1
(43) Pub. Date: Dec. 12, 2024

(54) IgG EPITOPE AND APPLICATIONS THEREOF AS A DRUG TARGET

(71) Applicant: GUANGZHOU SIG BIOPHARMACEUTICAL TECHNOLOGY CO., LTD., Guangzhou (CN)

(72) Inventors: Xiaoyan QIU, Beijing (CN); Jingshu TANG, Beijing (CN); Shenghua ZHANG, Beijing (CN); Wenhua JIANG, Beijing (CN); Zhiqiao HE, Beijing (CN)

(73) Assignee: GUANGZHOU SIG BIOPHARMACEUTICAL TECHNOLOGY CO., LTD., Guangzhou (CN)

(21) Appl. No.: 18/780,422

(22) Filed: Jul. 22, 2024

Related U.S. Application Data
(63) Continuation-in-part of application No. 16/969,189, filed on Aug. 11, 2020, now Pat. No. 12,077,602.

(30) Foreign Application Priority Data
Feb. 12, 2018 (CN) 201810146884.X
Apr. 13, 2018 (CN) 201810330585.1

(51) Int. Cl. C07K 16/06; A61K 47/64; C07K 16/42

(52) U.S. Cl. CPC C07K 16/06; A61K 47/643; C07K 16/4283; C07K 2317/41; C07K 2317/522; C07K 2317/55

(57) ABSTRACT

IgG epitope and applications thereof as a target are provided. The IgG epitope is the C_{H}1 domain of non-B cell-derived IgG, and there is N-glycosylated sialic acid modification at the Asn162 site of the domain. The realization of its antigen functions must depend on the sialylation of the site. The present invention further discloses the applications of the IgG epitope as a drug target in preparing drugs for diagnosis and/or treatment of epithelial tumors. In addition, our studies showed that this antigen depends on the sialylation of Asn162 site as a drug target, and the sialylation of this site must depend on sialyltransferase ST3GAL4, indicating that the enzyme can be used as a drug target for preparing tumor therapeutic drugs. Further, integrin β4 is co-expressed and co-localized with IgG containing the epitope. IgG can be used as a marker for preparing drugs for the auxiliary detection of epithelial tumors.

### CROSS REFERENCE TO THE RELATED APPLICATIONS

This application is the continuation-in-part of U.S. application Ser. No. 16/969,189 filed Aug. 11, 2020 which is the national phase entry of International Application No. PCT/CN2018/099279, filed on Aug. 8, 2018, which is based upon and claims priority to Chinese Patent Application No. 201810146884.X, filed on Feb. 12, 2018 and Chinese Patent Application No. 201810330585.1, filed on Apr. 13, 2018, the entire contents of which are incorporated by reference herein.

### SEQUENCE LISTING

The present application contains a Sequence Listing which has been submitted in xml format via EFS-Web and is hereby incorporated by reference in its entirety. Said xml copy is named GBBJGC001-CIP_Sequence_Listing_20240715.xml, created Jul. 15, 2024, and is 5,479 bytes in size.

### TECHNICAL FIELD

The present invention belongs to the general technical field of tumor diagnosis and treatment in immunology, and relates to a non-B cell-derived IgG, in particular to an IgG epitope and applications thereof as a drug target.

### BACKGROUND

Source and existing research of RP215 monoclonal antibody: In 1980s, in order to obtain monoclonal antibodies specifically recognizing ovarian cancer, the Lee research group of Columbia University in Canada immunized animals with proteins extracted from ovarian cancer cell lines and acquired nearly 3,000 hybridoma cells. They found a hybridoma cell could recognize ovarian cancer cells but not normal ovarian cells, called RP215, but they did not know which antigens it recognized at that time, so the antigen it recognized was temporarily named “CA215”. Later it was found that, RP215 not only recognized ovarian cancer well, but also showed good specificity in recognizing other types of tumor cells, thus CA215 was defined as “pan-cancer-marker”. In 2017, Lee research group identified CA215. They obtained a large amount of “CA215” from the culture supernatant of ovarian cancer cell lines using affinity chromatography. The 32 peptide fragments provided by mass spectrometry were all IgG heavy chains. In order to further confirm this result, they used the purified “CA215” as the antigen to prepare five strains of specific monoclonal antibodies. The experiments confirmed that all of the five monoclonal antibodies recognized IgG molecules. It proved that CA215 was IgG expressed by cancer cells. Subsequent results confirmed that the glycosylation of IgG expressed by cancer cells was significantly different from that of the circulating IgG. The epitope recognized by RP215 is the glycosyl-related epitope unique to this type of IgG heavy chain variable region (refer to Chinese Patent Publication No. 102901817A).

In other words, RP215 could not recognize IgG (circulating IgG) secreted by B lymphocytes after differentiation into plasma cells, but it could recognize IgG expressed by non-B cells. Studies have shown that the IgG recognized by RP215 (hereinafter referred to as RP215-IgG) can be expressed by cells of different lineages of cancers, but not or a few in normal non-B cells, so it is collectively referred to as non-B cell-derived IgG (non-B-IgG). Non-B-IgG is greatly different from the traditional B cell-derived IgG in the structure and functions.

RP215-IgG that is overexpressed by tumor stem-like cells, tends to be located at the junction between the cell surface and the cell, especially the focal adhesion structure. In addition, high-level RP215-IgG cells have a high adhesion capacity to the extracellular matrix (ECM), high migration and invasiveness in vitro and in vivo, to enhance the self-renewal and tumor formation ability of tumor stem cells. However, the specific structure and nature of the unique epitope recognized by RP215 and the mechanism of RP215-IgG-driven tumor occurrence and development are still unknown, so it is impossible to prepare the drugs for diagnosis or treatment specifically targeting IgG other than RP215.

Through our efforts, we have been clearly aware that, IgG that can be specifically recognized by RP215 is non-B cell-derived IgG, and the recognized site on the IgG is highly sialylated, and the sialylated glycosyl group is not related with O-glycosylation. This special sialylated IgG can be used as a marker to identify stem cells or progenitor cells, and then prepare related preparations based on the glycoprotein.

In view of the close correlation between non-B cell-derived IgG and epithelial cancer cells, further studies on the molecular structure of the IgG will facilitate the intervention of various malignant tumors.

### SUMMARY

The object of the present invention is to identify an epitope that can be used as a target through the further studies on the molecular structures and functions of non-B cell-derived IgG and provide new uses of the epitope.

Non-B cell-derived IgG was finally found through the related protease digestion analysis, glycosylation analysis and protein functional site analysis, and its tumor-related functions mainly depended on the sialylation of specific sites.

Based on our study results, the first aspect of the present invention provides an IgG epitope, which is the C_{H}1 domain of non-B cell-derived IgG, and has N-glycosylated sialic acid modification at Asn162 site of the domain.

Alternatively or preferably, the amino acid sequence of the foregoing IgG epitope is shown as SEQ ID NO: 1.

The second aspect of the present invention provides application of the IgG epitope in preparing drugs for diagnosis and/or treatment of tumors, and the tumors are epithelial tumors. The IgG containing the sialylated epitope at Asn162 specific site has been shown to promote tumor proliferation, migration and invasion ability.

Alternatively or preferably, in the above application, the tumors are non-small cell lung cancer, intestinal cancer, breast cancer, prostate cancer, kidney cancer, bladder cancer, saliva gland cystadenocarcinoma, gastric cancer, pancreatic cancer or esophageal cancer.

The third aspect of the present invention provides the application of the IgG epitope as a ligand of integrin α6β4 in preparing drugs for diagnosis and/or treatment of diseases mediated by α6β4-FAK-c-Met pathway. Studies have shown that IgG containing the epitope and integrin α6β4 bind to form a complex to promote the activation of integrin-FAK signaling pathway.

The fourth aspect of the present invention provides the application of sialyltransferase ST3GAL4 as a drug target in preparing tumor therapeutic drugs, and the tumors are epithelial tumors. The sialylation of the specific site containing the epitope depends on sialyltransferase ST3GAL4.

The present invention further provides the application of sialyltransferase ST3GAL4 combined with sialyltransferase ST3GAL6 as a drug target in preparing tumor therapeutic drugs, and the tumors are epithelial tumors. Studies have shown that sialyltransferase ST3GAL6 has an auxiliary role in the sialylation process of the epitope-specific site, therefore, the combination of them can be used as a drug target to intervene the epitope sialylation, thereby affecting its subsequent functions.

The fourth aspect of the present invention further provides the application of integrin β4 as a marker in preparing drugs for auxiliary detection of epithelial tumors. IgG containing the epitope and integrin β4 are co-expressed and co-localized at the tissue level and the cellular level, therefore, when integrin β4 is detected, equivalently, IgG is detected and epithelial tumors are detected.

### An Immunogenic Peptide

The present disclosure also provides an isolated immunogenic peptide, wherein the immunogenic peptide comprises an amino acid sequence as shown in SEQ ID NO:1 with a N-glycosylated sialic acid modification at Asn162 site;

wherein the CHI domain of RP215-IgG is as shown below:

(SEQ ID NO: 1)
Lys Asp Tyr Phe Pro Glu Pro Val Thr Val Ser Trp
Asn Ser Gly Ala Leu Thr Ser Gly Val His
Thr Phe Pro Ala Val Leu Gln Ser Ser Gly Leu Tyr
Ser Leu Ser Ser Val Val Thr Val Pro Ser Ser
Asn Phe Gly Thr Gln Thr Tyr Thr Cys Asn Val Asp
His Lys Pro Ser Asn Thr Lys Val Asp Lys
Thr Val Glu Arg Lys Cys Cys Val Glu Cys Pro Pro
Cys Pro Ala Pro Pro Val Ala Gly Pro Ser Val
Phe Leu Phe Pro Pro Lys Pro Lys Asp Thr Leu Met
Ile Ser Arg Thr Pro Glu Val Thr Cys Val Val
Val Asp Val Ser His Glu Asp Pro Glu Val Gln Phe
Asn Trp Tyr Val Asp Gly Val Glu Val His Asn
Ala Lys Thr Lys Pro Arg Glu Glu Gln Phe Asn Ser
Thr Phe Arg Val Val Ser Val Leu Thr Val Val
His Gln Asp Trp Leu Asn Gly Lys Glu Tyr Lys;
Note:
the Asn in bold is the 162 site modified with N-glycosylated sialic acid.

In the present disclosure, the site “162” refers to the 162^{nd} position of the constant region of RP215-IgG heavy chain under the Kabat Numbering Criteria, which corresponds to the 13^{th} natural order of SEQ ID No. 1.

The “isolated” refers to material substantially or essentially free from components that normally accompany it as found in its native state. Thus, the immunogenic peptide provided herein do not contain materials normally associated with their in situ environment. Typically, the isolated immunogenic peptide described herein are at least about 80% pure, usually at least about 90% pure, and preferably at least about 95% pure as measured by intensity on a stained gel or by other method known in the art. As an example, protein purity or homogeneity may be indicated by methods well known in the art, such as polyacrylamide gel electrophoresis of a protein sample, followed by visualization upon staining. For certain purposes high resolution will be needed, and HPLC, affinity chromatography or similar means for purification utilized.

As used herein, the term “immunogenic peptide” refers to proteins, peptides, and polypeptides that are immunologically active in the sense that once administered to the host (animal or human), it is able to evoke an immune response of the humoral and/or cellular type directed against the peptide.

The immunogenic peptides provided herein can be peptides, portions or fragments of proteins, in particular peptides, portions or fragments of RP215-IgG.

The immunogenic peptides provided herein are served as immunogen to induce a specific immune response in a host.

As used herein, the term “immunogen” means a substance that induces a specific immune response in a host animal. The immunogen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof.

An immunogen generally encompasses any immunogenic substance, i.e., any substance that elicits an immune response (e.g., the production of specific antibody) when introduced into animal or human, and that is capable of specifically binding to an antibody that is produced in response to the introduction of the immunogen.

An immunogen may optionally be administered in, or with, one or more adjuvants.

In some embodiments of the isolated immunogenic peptide, the isolated immunogenic peptide is C_{H}1 domain of RP215-IgG with a N-glycosylated sialic acid modification at Asn162 site.

In some embodiments of the isolated immunogenic peptide, the isolated immunogenic peptide is Fab fragment of RP215-IgG with a N-glycosylated sialic acid modification at Asn162 site.

Fab (fragment antigen-binding) fragments are the antibody binding regions of an antibody. It contains one complete light chain in its entirety and the Variable and C_{H}1 portion of one heavy chain. The Fab can be further divided into a variable fragment (Fv) composed of the VH and VL domains, and a constant fragment (Fb) composed of the CL and C_{H}1 domains.

In some embodiments of the isolated immunogenic peptide, the isolated immunogenic peptide is RP215-IgG itself with a N-glycosylated sialic acid modification at Asn162 site.

An immunogenic conjugate Peptide-carrier coupling is often used to prepare antibodies, since individual peptides are usually too small in size to trigger sufficient immune responses. Carrier proteins are beneficial for stimulating helper T cells and further inducing B cell immune responses.

Therefore, the present disclosure also provides an immunogenic conjugate, comprising:
1) the isolated immunogenic peptide provided herein; and
2) carrier.

The carrier is conjugated or otherwise covalently or non-covalently bound to the immunogenic peptide.

The carrier is conjugated to the immunogenic peptide at N-terminus or C-terminus.

The term “carrier” as used herein means a structure in which a immunogen or a immunogenic peptide can be incorporated into or associated with, thereby presenting or exposing the immunogen or part of the immunogenic peptide to the immune system of a human or animal. The “carrier” further comprises methods of delivery wherein immunogenic peptides may be transported to desired sites by delivery mechanisms.

The term “carrier” further includes those known to the skilled persons in the art including, but not limited to, keyhole limpet hemocyanin (KLH), bovine serum albumin (BSA), OVA, maltose binding protein (MBP), flagellin, thyroglobulin, polymers of D- and/or L-amino acids, tetanus toxoid.

In some particular embodiments, the immunogenic conjugate provided herein comprising:
the immunogenic peptide as shown in SEQ ID NO:1 with a N-glycosylated sialic acid modification at Asn162 site; and BSA;
the BSA is conjugated to the isolated immunogenic peptide at N-terminus or C-terminus.

The immunogenic conjugate provided herein is served as immunogen to induce a specific immune response in a host.

### An Immunogenic Composition

The present disclosure also provides an immunogenic composition, comprising:
an effective amount of the isolated immunogenic peptide provided herein or the immunogenic conjugate provided herein; and
a pharmaceutically acceptable excipient.

It is also to be understood that the immunogenic compositions provided herein can include pharmaceutically acceptable excipient such as adjuvants, preservatives, diluents, emulsifiers, stabilizers, and other components that are known and used in vaccines. Any adjuvant system known in the art can be used in the compositions described herein. Such adjuvants include, but are not limited to, Freund's incomplete adjuvant, Freund's complete adjuvant, polydispersed β-(1,4) linked acetylated mannan, polyoxyethylene-polyoxypropylene copolymer adjuvants, modified lipid adjuvants, saponin derivative adjuvants, large polymeric anions such as dextran sulfate, and inorganic gels such as alum, aluminum hydroxide, or aluminum phosphate.

### Applications of the Isolated Immunogenic Peptide

The present disclosure provides a method for stimulating or inducing the immune system of host (animal or human) to produce immune response. Such a method generally involves at least the step of administering to the host an immunologically-effective amount of one or more of the isolated immunogenic peptide or the immunogenic conjugate or the immunogenic composition to the host in an amount and for a time sufficient to stimulate the immune system. In the practice of the method, administration of the composition preferably induces a detectable amount of antibodies in the host.

The present disclosure also provides a method for an immune response comprising:
administering to a host (non-human animal or a human) an immunologically-effective amount or effective amount of the isolated immunogenic peptide or the immunogenic conjugate or the immunogenic composition provided herein.

The term “immunogenically-effective amount” or “effective amount” has its usual meaning in the art, i.e., an amount of an immunogen that is capable of inducing an immune response. The “effective amount” is readily determined by one of skill in the art following routine procedures.

It will be desirable to deliver the disclosed immunogenic peptide, immunogenic conjugate or immunogenic composition in suitably-formulated pharmaceutical vehicles by one or more standard delivery routes, including, for example, subcutaneously, intraocularly, intravitreally, parenterally, intravenously, intracerebroventricularly, intramuscularly, intrathecally, orally, intraperitoneally, transdermally, topically, orally, or nasal inhalation, or by direct injection to one or more cells, tissues, or organs.

For administration of an injectable aqueous solution, for example, the solution may be suitably buffered, if necessary, and the liquid diluent rendered isotonic with sufficient saline or glucose. These particular aqueous solutions are suitable for intravenous, intramuscular, subcutaneous, and intraperitoneal administration. A sterile aqueous medium that can be employed will be known to those of ordinary skill in the art in light of the present disclosure. The person responsible for administration will determine the appropriate dose for the individual subject. Moreover, for human administration, preparations should meet sterility, pyrogenicity and the general safety and purity standards as required. Sterile injectable compositions may be prepared by incorporating the disclosed immunogenic compositions in the required amount in the appropriate solvent with several of the other ingredients enumerated above, as required, followed by filtered sterilization.

The present disclosure also provides a method of producing an antibody comprising: administering to a non-human or a human an effective amount of the isolated immunogenic peptide or the immunogenic conjugate or the immunogenic composition provided herein.

In some embodiments, the antibody is a monoclonal antibody or polyclonal antibody.

The immunogenic polypeptide or immunogenic conjugate or immunogenic composition of the present disclosure find particular utility in the production of antibodies specific for the epitope given herein (C_{H}1 domain of RP215-IgG with a N-glycosylated sialic acid modification at Asn162 site). Antibodies specific for the epitope are useful, e.g., in both diagnostic and therapeutic purposes.

Antibodies specific for the epitope of the present disclosure can be generated by methods well known in the art. Such antibodies can include, but are not limited to, polyclonal, monoclonal, chimeric, humanized, single chain, Fab fragments etc. Numerous methods for producing polyclonal and monoclonal antibodies are known to those of skill in the art, and can be adapted to produce antibodies specific for the epitope (see, e.g., Coligan Current Protocols in Immunology Wiley/Greene, NY; Paul (ed.) (1991); (1998) Fundamental Immunology Fourth Edition, Lippincott-Raven, Lippincott Williams & Wilkins; Harlow and Lane (1989) Antibodies: A Laboratory Manual, Cold Spring Harbor Press, NY, USA; Stites et al. (Eds.) Basic and Clinical Immunology (4th ed.) Lange Medical Publications, Los Altos, Calif., USA and references cited therein; Goding, Monoclonal Antibodies: Principles and Practice (2d ed.) Academic Press, New York, N.Y., USA; 1986; and Kohler and Milstein (1975).

Briefly, a polyclonal antibody is prepared by immunizing the host with the immunogenic polypeptide or immunogenic conjugate or immunogenic composition in accordance with the present disclosure and collecting antisera from that immunized host. A wide range of host species can be used for the production of antisera. Typically the animal used for production of anti-antisera is a rabbit, a mouse, a rat, a hamster, a guinea pig or a goat.

MAbs may be readily prepared through use of well-known techniques. Typically, this technique involves immunizing a suitable host with the immunogenic polypeptide or immunogenic conjugate or immunogenic composition. As an example, the use of mice are preferred, with the BALB/c mouse being preferred as this is routinely used and generally gives a higher percentage of stable fusions. The antibody-producing B lymphocytes from the immunized animal are then fused with cells of an immortal myeloma cell, generally one of the same species as the animal that was immunized.

Any one of a number of myeloma cells may be used, as are known to those of skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

Methods for generating hybrids of antibody-producing spleen or lymph node cells and myeloma cells usually comprise mixing somatic cells with myeloma cells in a ratio from about 20:1 to about 1:1, in the presence of agent(s) that promote the fusion of cell membranes.

Fusion procedures usually produce viable hybrids. The viable fused hybrids are differentiated from the parental, unfused cells by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells, followed by testing the clonal supernatants for the desired reactivity, such as radioimmunoassays, enzyme immunoassays, cytotoxicity assays, plaque assays, dot immunobinding assays, and the like. The selected hybridomas would then be serially diluted and cloned into individual antibody-producing cell lines, which clones can then be propagated indefinitely to provide MAbs.

The present disclosure also provides an antibody, which is obtained by the method provided herein. In particular, selected antibody is specific for the C_{H}1 domain of RP215-IgG with a N-glycosylated sialic acid modification at Asn162 site.

According to the above technical solutions provided herein, the present invention has the following beneficial effects:

The present invention has identified the functional molecular structure of non-B cell-derived IgG, which is a highly sialylated structure of N-glycan at the Asn162 site on the C_{H}1 domain of IgG. It is over-expressed in stem cells of epithelial tumors and is very important to the carcinogenic properties of a variety of epithelial malignancies; in addition, it is an attractive target for antibody therapy, Car-T cell therapy and small molecule compounds, which provides an effective drug target for the treatment of related cancers and an epitope for antibody drug research.

### BRIEF DESCRIPTION OF THE DRAWINGS

FIG. 1-1A: Schematic diagram of IgG recognized by RP215 enriched from lung squamous cell carcinoma using ProteinG combined with RP215-CNBr affinity chromatography column in Embodiment 1;

FIG. 1-1B: Structural diagram of each carbohydrate chain and the content of different glycans in Embodiment 1;

FIG. 1-1C: SNA and MAL I that recognized sialic acid were used to identify the status of IgG sialic acid in each component of affinity chromatography column in Embodiment 1;

FIG. 1-1D: Western blot results of sialylated IgG after digested with N-glycosidase, O-glycosidase and sialidase, respectively. Elution: eluent; buffer1: digestion buffer of N-glycosidase and O-glycosidase; buffer2: digestion buffer of sialidase in Embodiment 1.

FIG. 1-2A: Amino acid sequences of unmutated RP215-IgG (WT) (SEQ ID NO: 1) and two mutant (C_{H}1mu) (SEQ ID NO: 2) and (C_{H}2mu) (SEQ ID NO: 3), the framed part is the corresponding mutation site in Embodiment 1.

FIG. 1-2B: Detection of the recognition of RP215 by Western blot after over-expressing RP215-IgG (the variable region sequence is VH5-51/D3-9/JH4) and the corresponding mutant in 293T cells in Embodiment 1;

FIG. 1-2C: Detection of the recognition of RP215 by Western blot after overexpressing RP215-IgG (the variable region sequence is V_{H}5-51/D3-9/J_{H}4) and the corresponding mutant in NCI-H520 cells; where, mock is a negative control and vector is an empty vector in Embodiment 1.

FIG. 2-1A: Western blots to detect the expression of RP215-IgG in non-small cell lung cancer cell lines, GAPDH is the internal reference in Embodiment 2;

FIG. 2-1B: Western blots to detect the secretion of RP215-IgG in the culture supernatant of NCI-H520 cells and SK-MES-1 cells in Embodiment 2;

FIG. 2-1C: Immunofluorescence method to detect the localization of RP215-IgG in NCI-H520 cells and SK-MES-1 cells, Hochest labeled nuclei, secondary antibody: goat anti-mouse Alexa Flour 488, Scale 25 μm in Embodiment 2;

FIG. 2-1D: Flow cytometry to detect the expression of RP215-IgG on the cell membrane of non-small cell lung cancer cell line (live cell staining), secondary antibody: goat anti-mouse Alexa Flour 488 in Embodiment 2.

FIG. 2-2A: Western blot detection of knockdown after NCI-H520 cells are transfected with control or siRNA against IgG, respectively. GAPDH is internal reference in Embodiment 2;

FIG. 2-2B: NCI-H520 cells are transfected with siRNA for 36 h, then Transwell assay is performed to detect cell migration ability in Embodiment 2;

FIG. 2-2C: NCI-H520 cells are transfected with siRNA for 36 h, then Matrigel-Transwell assay is performed to detect the cell invasion ability in Embodiment 2;

FIG. 2-2D: Western blot detect the result of knockdown after knockdown of IgG in SK-MES-1 with IgG siRNA, GAPDH is internal reference;

FIG. 2-2E: SK-MES-1 cells are transfected with siRNA for 36 h, then Transwell assay was conducted to detect the cell migration ability in Embodiment 2;

FIG. 2-2F: SK-MES-1 cells are transfected with siRNA for 36 h, and then Matrigel-Transwell assay was conducted to detect the invasive ability. *, P<0.05; **, P<0.01; scale 200 μm.

FIG. 2-3A: Colony formation assay was performed to detect cell proliferation and self-renewal after knockdown of IgG for NCI-H520 cells in Embodiment 2;

FIG. 2-3B: Colony formation assay was performed to detect cell proliferation and self-renewal after knockdown of IgG for SK-MES-1 cells in Embodiment 2. **, P<0.01.

FIG. 2-4A: Western blot to detect RP215 recognition, commercial anti-human IgG is a negative control, GAPDH is an internal reference in Embodiment 2;

FIG. 2-4 B: Plate clonality assay results in Embodiment 2;

FIG. 2-4 C: Transwell assay to detect the cell migration ability in Embodiment 2;

FIG. 2-4D: Matrigel-Transwell assay to detect cell invasion ability. WT: Wild type IgG, C_{H}1mu: C_{H}1 mutant IgG; Vector: control empty vector; mock: negative control. ns, not significant; *, P<0.05; **, P<0.01; ***, P<0.001 in Embodiment 2.

FIG. 2-5A: Pictures of tumor volumes in WT, C_{H}1mu and Vector groups at the end of the experiment in Embodiment 2;

FIG. 2-5B: Growth curve of tumor volumes in WT, C_{H}1mu and Vector groups in Embodiment 2;

FIG. 2-5C: Tumor volumes graph in WT, C_{H}1mu and Vector groups at the end of the experiment in Embodiment 2;

FIG. 2-5D: Tumor weights graph in WT, C_{H}1mu and Vector group at the end of the experiment in Embodiment 2. ns, not significant; **, P<0.01; ***, P<0.001.

FIG. 3-1A: The result of RP215 recognition after silence of four sialyltransferases in Embodiment 3;

FIG. 3-1B: The result of RP215 recognition after overexpression of four sialyltransferases, of which, GAPDH is an internal reference in Embodiment 3.

FIG. 3-2 shows the immunohistochemical detection results of RP215, anti-ST3GAL4 and anti-ST3GAL6 antibodies in lung adenocarcinoma and lung squamous cell carcinoma tissues in Embodiment 3.

FIG. 4-1A: Gene Ontology analysis results of RP215-IgG specific interacting protein in Embodiment 4;

FIG. 4-1B: GO term analysis method to analyze proteins with high score of MS peptide segments in Embodiment 4.

FIG. 4-2A: Western blot results of integrin β1, integrin β4, integrin α6 antibody and RP215 after RP215 and NCI-H520 cell lysate are incubated for immunoprecipitation in Embodiment 4;

FIG. 4-2B: Western blot results of integrin β4 antibody and RP215 after integrin β4 antibody and NCI-H520 cell lysate are incubated for immunoprecipitation in Embodiment 4;

FIG. 4-2C: Western blot results of integrin α6 antibody and RP215 after integrin α6 antibody and NCI-H520 cell lysate are incubated for immunoprecipitation. mIgG is a control antibody, rIgG is a rabbit antibody, as a co-IP control in Embodiment 4.

FIG. 4-3 shows the immunohistochemical detection of staining and localization of RP215-IgG and integrin β4 in lung squamous cell carcinoma tissues, and the adjacent squamous cell carcinoma tissue sections showed similar staining patterns of RP215 and integrin β4 antibody in Embodiment 4. Scale, 50 μm.

FIG. 4-4A: Flow cytometry to analyze the expression levels of RP215-IgG and integrin β4 on the cell surface of lung squamous cell carcinoma PDX models in Embodiment 4. The analysis strategy: firstly, cell debris is excluded by cell size and particle size gate, live cells are obtained by 7-AAD negative cell gate, then the RP215-IgG positive and RP215-IgG negative cell populations are obtained by RP215-FITC gate; and then the integrin β4 expression levels of the above two cell populations are analyzed by anti-integrin β4-PE;

FIG. 4-4B: Flow cytometry to sort out NCI-H520 cells with strong positive and weak positive RP215, and Western blots to detect the expression level of integrin β4 in the cells with high expression of RP215-IgG (RP215-IgG^{high}) and low expression of RP215-IgG (RP215-IgG^{low}) in Embodiment 4.

FIG. 4-5 shows the immunofluorescence analysis of the location of RP215-CIgG (first column from the left) and integrin β4 (second column from the left) in NCI-H520 cells in Embodiment 4. Scale, 10 μm.

FIG. 4-6A: NCI-H520 cells are transfected with control or siRNA against IgG, respectively in Embodiment 4. Western blots are used to detect knockdown effects and molecules related to the Integrin-FAK signaling pathway. GAPDH is an internal reference.

FIG. 4-6B: SK-MES-1 cells are transfected with control or siRNA against IgG, respectively in Embodiment 4. Western blots are used to detect knockdown effects and molecules related to the Integrin-FAK signaling pathway. GAPDH is internal reference.

FIG. 4-7 shows partial mass spectrometry scoring result of RP215-IgG specific interacting proteins in Embodiment 4.

FIG. 4-8A: Statistical results of straining after NCI-H520 cells are transfected with control or siRNA against IgG, respectively, and incubated in cell lysate and RTK phosphorylation chips in Embodiment 4;

FIG. 4-8B: Statistical results of straining after SK-MES-1 cells are transfected with control or siRNA against IgG, respectively, and incubated in cell lysate and RTK phosphorylation chips in Embodiment 4.

FIG. 4-9A: NCI-H520 cells are transfected with control or siRNA against IgG, respectively. Western blots are used to detect knockdown effect and c-Met phosphorylation level and related molecules of downstream signaling pathway. GAPDH is an internal reference in Embodiment 4.

FIG. 4-9B: SK-MES-1 cells are transfected with control or siRNA against IgG, respectively. Western blots are used to detect the knockdown effect and c-Met phosphorylation level and related molecules of downstream signaling pathways in Embodiment 4. GAPDH is an internal reference.

FIG. 4-10A: RP215 and c-Met antibody are incubated with NCI-H520 cell lysate for immunoprecipitation, and Western blots detection is performed with c-Met antibody and RP215 in Embodiment 4;

FIG. 4-10B: NCI-H520 cells are transfected with control siRNA and c-Met siRNA, respectively, and Western blots are used to detect the knockdown effect in Embodiment 4. RP215 is incubated with NCI-H520 cell lysate transfected with control siRNA and c-Met siRNA, respectively, and Western blots detection is performed with integrin β4 antibody and RP215;

FIG. 4-10C: The knockdown effect detected by Western blots after NCI-H520 cells are transfected with control siRNA and integrin β4 siRNA in Embodiment 4. Antibody RP215 is incubated with NCI-H520 cell lysate transfected with control siRNA and integrin β4 siRNA, respectively, and Western blots detection is performed with c-Met antibody and RP215.

FIG. 4-11A: The experimental results on the inhibition of Integrin-FAK signaling pathway of NCI-H520 cells after exogenous addition of antibody RP215 in Embodiment 4. Add control antibody mIgG (50 μg/ml) or different concentrations of antibody RP215 (2 μg/ml, 10 μg/ml, 50 μg/ml) in the cell culture supernatant of NCI-H520. Cells are collected at 12 h, 24 h and 36 h, and Integrin-FAK signaling pathway is detected by Western blots;

FIG. 4-11B: The experimental results on the inhibition of clone formation ability of NCI-H520 cells after exogenous addition of antibody RP215 in Embodiment 4. Add control antibody mIgG (50 μg/ml) or different concentrations of antibody RP215 (2 μg/ml, 10 μg/ml, 50 μg/ml) in the cell culture supernatant of NCI-1520. Colony formation ability is detected by colony forming assay. ns, not significant; ***, P<0.001.

FIG. 4-12A: The experimental results on the inhibition of Integrin-FAK signaling pathway of SK-MES-1 cells after exogenous addition of RP215-IgG reversible antibody RP215 in Embodiment 4. SK-MES-1 cell culture supernatant is added with antibody RP215 (10 μg/ml) and different concentrations of RP215-CIgG (2 μg/ml, 10 μg/ml, 50 μg/ml) or flow-through liquid components (10 μg/ml, 50 μg/ml). The cells are collected at 48 h, and Western blot is performed to detect the Integrin-FAK signaling pathway.

FIG. 4-12B: The experimental results on the inhibition of colony formation ability of SK-MES-1 cells after exogenous addition of RP215-IgG reversible antibody RP215 in Embodiment 4. SK-MES-1 cell culture supernatant is added with antibody RP215 (10 μg/ml) and different concentrations of RP215-CIgG (2 μg/ml, 10 μg/ml, 50 μg/ml) or flow-through liquid components (10 μg/ml, 50 μg/ml). The culture results and statistical results of colony formation ability detected by colony formation assay. ns, not significant; ***, P<0.001,

FIG. 5A: Immunohistochemical staining score results in 242 lung cancer tissues in Embodiment 5;

FIG. 5B: Immunohistochemical staining sections of RP215-IgG in tissues of patients with different types of lung cancer in Embodiment 5;

FIG. 5C: RP215 immunohistochemical staining results of normal alveolar tissues (Alveolus), drainage lymph node tissues (Lymph node) and bronchial tissues (Bronchus) in Embodiment 5;

FIG. 5D: Kaplan-Meier survival curve analysis of the correlation between RP215 staining score grade and 5-7 year survival rate of patients with lung squamous cell carcinoma in Embodiment 5.

FIG. 6A: Schematic diagram of the experimental procedure of antitumor in vivo experiment using RP215 in Embodiment 7;

FIG. 6B: Tumor changes after injecting RP215 (monoclonal antibody) and control mIgG, respectively in Embodiment 7;

FIG. 6C: Changes of tumor volume with time in the PDX tumor models in Embodiment 7;

FIG. 6D: Scatter plot and line chart of tumor weight changes in different days in the first group in Embodiment 7.

FIG. 6E: Scatter plot and line chart of tumor weight changes in different days in the second group in Embodiment 7.

FIG. 7A: Schematic diagram of radiolabeling of ¹²⁴I-RP215 in Embodiment 7;

FIG. 7B: Radio-TLC analysis results of ¹²⁴I-RP215 before and after labeling and purification in Embodiment 7;

FIG. 7C: Micro-PET/CT imaging of ¹²⁴I-RP215 at different times in Embodiment 7.

FIG. 8: The antibody titer of the generated antibodies for the antigen.

FIG. 9: Comparison of antibody titers generated with three different immunogens. The x-axis represents serum dilution, with 1 being 1:500, 2 being 1:1000, 3 being 1:2000, 4 being 1:4000, 5 being 1:8000, and 6 being 1:16000. The y-axis represents the OD450 value.

### DETAILED DESCRIPTION OF THE EMBODIMENTS

In the following embodiments, RP215-IgG refers to IgG that can be specifically recognized by monoclonal antibody RP215, and it is non-B cell-derived IgG.

### Sources of Biological Materials:

### Monoclonal Antibody RP215:

For hybridoma cell line, RP215-containing ascites was produced by culturing hybridoma clones in the abdominal cavity of BALB/c mice sensitized with Freund's adjuvant. According to the manufacturer's description, antibodies were purified from ascites using protein G affinity chromatography (GE healthcare, USA), then concentrated and obtained using PBS as a solvent.

### Cancer Cell Lines:

The LSCC cell lines NCI-H520, SK-MES-1 and 293T were obtained from American Type Culture Collection (ATCC, Manassas, VA, USA) and kept by the Center for Human Genomics, Peking University.

### Example 1 Functional Structure Determination of RP215-IgG

1. Purification of IgG from Cancer Tissues
(1) Tumor IgG was purified from the PDX tumor models established by non-small cell lung cancer tissues, breast cancer or ovarian cancer tissues, or lung squamous cell carcinoma (LSCC) tissues in advance using protein G-Sepharose 4 Fast Flow (GE healthcare, USA). The use of PDX tumor models was to exclude the effect of IgG derived from peripheral blood.
(2) Purification of RP215-IgG with RP215 affinity column.

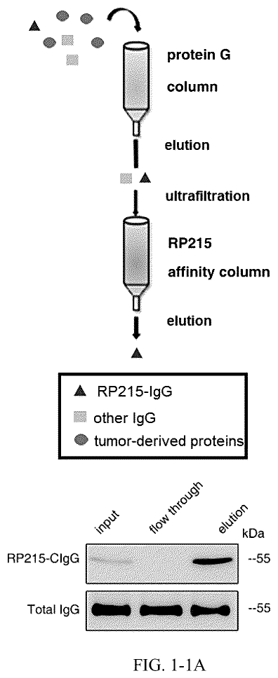

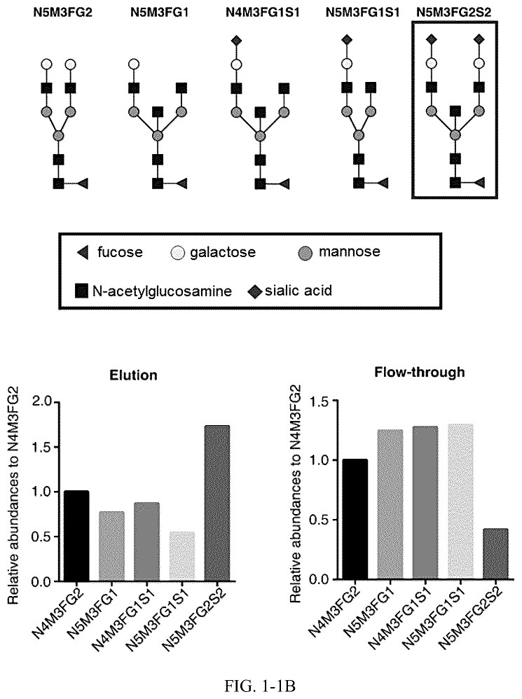

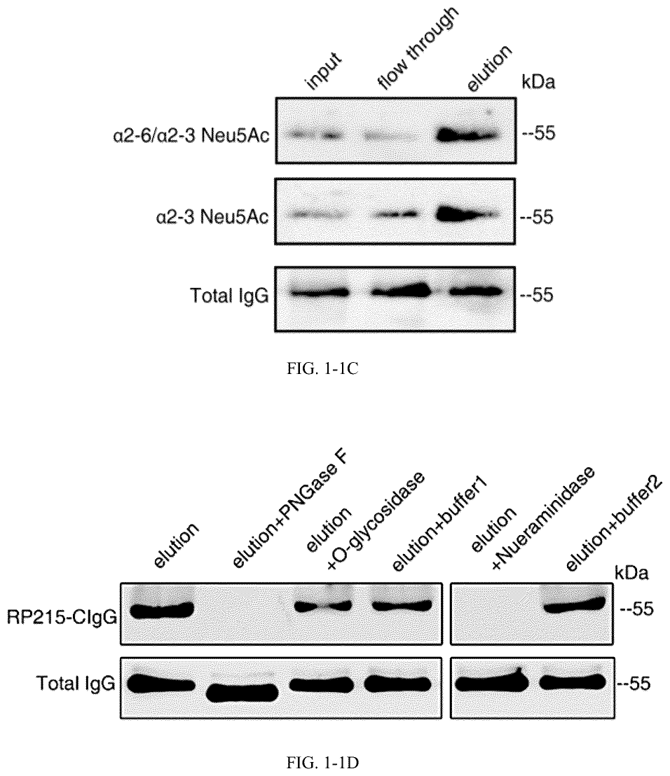

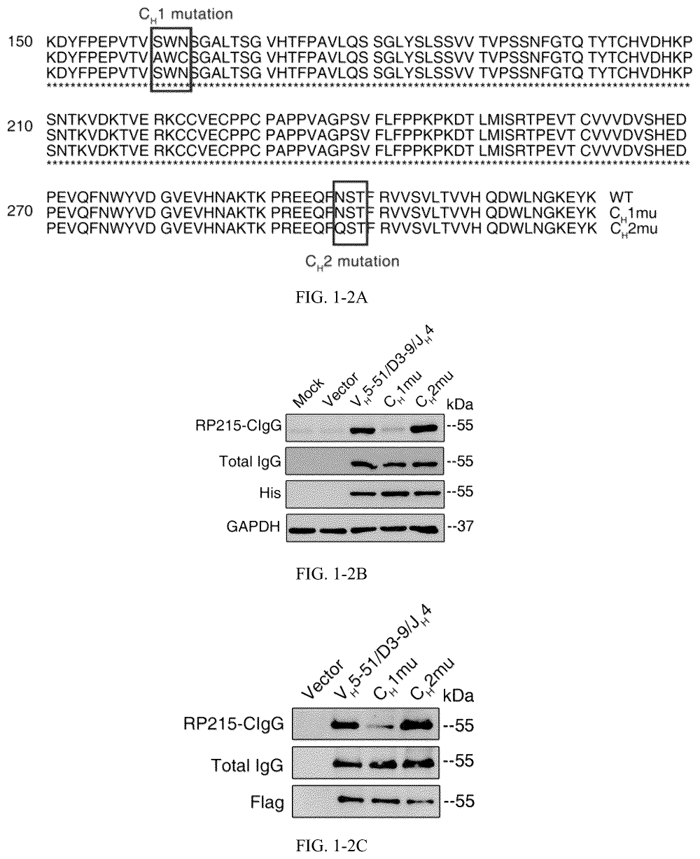

Preparation of RP215 affinity column: the monoclonal antibody RP215 was coupled with Sepharose 4 Fast Flow (GE Healthcare, USA) activated by CNBr, as shown in FIG. 1-1A. The operations were performed according to the instructions. (1) After 330 mg CNBr-sepharese 4B was activated with 1 mM hydrochloric acid, it was equilibrated with coupling buffer (0.1M NaHCO₃, 0.5M NaCl, pH8.3). (2) 5 mg of RP215 antibody was dissolved in coupling buffer, then added into the activated CNBr agarose gel filler and incubated overnight at 4° C. (3) At 4° C., the coupled gel was washed with Tris-HCl (pH 8.0, 0.1 M) and resuspended overnight to block the unconjugated activation site. (4) The affinity column was washed alternately with acidic washing solution (0.1M NaAc, 0.5M NaCl, pH 4.0) and alkaline washing solution (0.1M Tris, 0.5M NaCl, pH 8.0) at least 3 times to remove excessive RP215.

Purification of RP215-IgG with RP215 affinity columns: about 5 mg of tumor IgG that was adjusted to 1 μg/l in PBS was incubated with the affinity column at 4° C. overnight to bind RP215 on the affinity column to tumor IgG. \textcircled{6} After washing with at least 5 column volumes of PBS, elution procedure was performed using 0.1 M Tris-glycine (pH 2.4). The eluate was collected and concentrated with PBS ultrafiltration for further analysis. The concentrated eluate contained the separated IgG, which was labeled RP215-IgG.

### 2. IgG Glycosylation Analysis

### Release of N-Glycan

50 μg of RP215-IgG, 2.5 μL of 200 mM DTT and 150 μL of 20 mM ammonium bicarbonate buffer were added to an ultrafiltration reactor (PALL, USA), incubated at 50° C. for 1 hour, then added with 10 μl of 200 mM IAA to the solution. The mixture was incubated at room temperature for 45 minutes in the darkness to denature the protein. The denatured protein was washed twice with 200 μl of 20 mM ammonium bicarbonate buffer. 1 μl of PNGase F and 200 μl of 20 mM ammonium bicarbonate buffer were added to the reactor. Then the mixture was further incubated at 37° C. overnight to achieve complex release of N-glycan.

After centrifugation, the released solution containing N-glycan was collected. Prior to derivatization, the solution was lyophilized.

### UPLC-HRMS Analysis of N-Glycan:

Prior to UPLC-HRMS analysis, the collected N-glycan was added to the derivatization solution. The derivatization solution included 10 μl aqueous solution containing 1 μl 10% acetic acid, 7 μl of isopropanol containing 30 mg/ml of 2,4-bis(diethylamino)-6-hydrazino-1,3,5-triazine and 2 μl of water. The derivatization reaction was completed at 37° C. for 2 hours.

The derivatized products were further analyzed directly by UPLC-Orbitrap (Thermo Fisher Scientific, Bremen, Germany) without any further purification. The mobile phase A was a 10 mM ammonium formate aqueous solution. The mobile phase B was acetonitrile. The mobile phase A increased from 20% to 50% within 15 minutes, holding for 5 minutes. Then mobile phase A decreased to 20% within 5 minutes, holding for 5 minutes. The flow rate was 0.4 ml/min, the column temperature was 10° C., and the injection volume was 3 l1. The ESI voltage was set to 3.2 kV for quality data collection, and 35 arb sheath gases and 10 arb auxiliary gases were applied to stabilize the ESI. Derivatized oligosaccharides were detected in a positive mode. The full scan quality range was from 800 to 3000 m/z.

The results showed that 28 types of N-glycan were detected, including fucose and sialic acid that were not commonly found in circulating IgG. Four types of glycans: N5M3FG2 and N5M3FG1 (both without sialic acid), and N4M3FG1S1 and N5M3FG1S1 (both containing a terminal sialic acid residue) had a higher unbound component in RP215, while the ratio of N5M3FG2S2 (with a sialylation biantennary structure) in the unbound components of RP215 was significantly reduced (FIG. 1-1B). In contrast, N5M3FG2S2 had a higher abundance in the RP215 binding components (FIG. 1-1B).

### 3. Analysis of IgG Epitope Sites

### Glycosylation Analysis of Recognition Sites:

The purified RP215-IgG was deglycosylated.

The eluent in the above section 1 (containing RP215-IgG, labeled as elution) was taken as the sample. In order to digest N-linked and O-linked glycans, the sample was added to the denaturation buffer and denatured at 100° C. for 10 minutes. Then, the above mixture was incubated in G7 reaction buffer containing NP-40 and an appropriate amount of glycosidase for 2 hours at 37° C. to digest the protein.

Glycosidase was N-glycosidase (PNGase F) (NEB, USA). PNGase F could hydrolyze almost all N-carbohydrate chain; O-glycosidase (NEB, USA) could hydrolyze O-carbohydrate chain;

In order to digest sialic acid, the samples were digested in G1 reaction buffer containing nueraminidase (Sialidase) (NEB, USA) for 2 hours at 37° C.

The experimental results were shown in FIG. 1-1D. After treatment with PNGaseF and neuraminidase, the band recognized by RP215 disappeared, and RP215 recognition was not disturbed after digestion with O-glycosidase. In addition, we found that the molecular weight of all N-linked carbohydrates between the innermost GlcNAc and Asn residues of RP215-IgG decreased from 55 kD to 50 kD, while IgG treated with O-glycosidase remained unchanged. The above results indicated that the epitope recognized by RP215 was related to the sialic acid residue of N-glycan on IgG.

FIG. 1-1A electropherogram showed that the purified elution product(containing the target IgG) could be recognized by RP215, and the input band of sample solution that did not pass through column was slightly displayed, while the flow-through liquid could not be recognized by RP215.

FIG. 1-1C showed the test results of identifying whether the RP215 affinity column could enrich sialylation IgG using lectins that could specifically recognize sialic acid-Sambucusnigra agglutinin (SNA) that mainly recognized sialic acid linked by α2,6, Maackiaamurensis leukoagglutinin I (MAL I) that mainly recognized sialic acid linked by α2,3. Results showed that the eluent contained sialic acid linked by a 2, 6 and a 2,3.

### 4. Analysis of Sialylation Recognition Sites of RP215-IgG

The Fab fragment of IgG is composed of C_{H}1 and the variable region. The variable region shows a great diversity. We assume that the possible N-glycan sites recognized by RP215 may be located in C_{H}1. According to our previous studies, RP215 can recognize IgG from different tissues of epithelial cancer that has different VDJ recombination patterns, so it is speculated that the epitope recognized by RP215 is not on the variable region, but should be on C_{H}1. Therefore, the atypical glycosylation motifs TVSWN¹⁶²SGAL (SEQ ID NO: 4) (S160A and N162C) found in the C_{H}1 domain were introduced with site-specific mutations for preliminary exploration. At the same time, the classic glycosylation site asparagine (N) 297 in the C_{H}2 domain was also introduced, which was replaced with glutamine (Q) as a control.

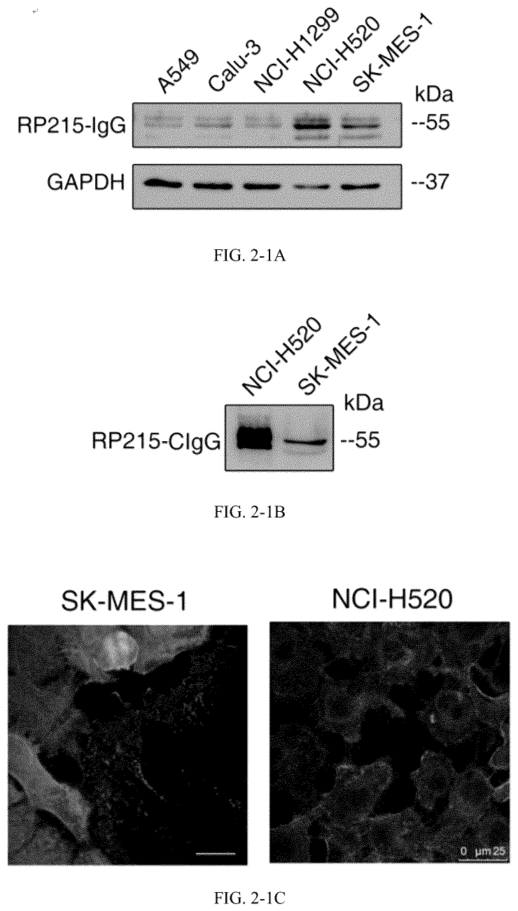

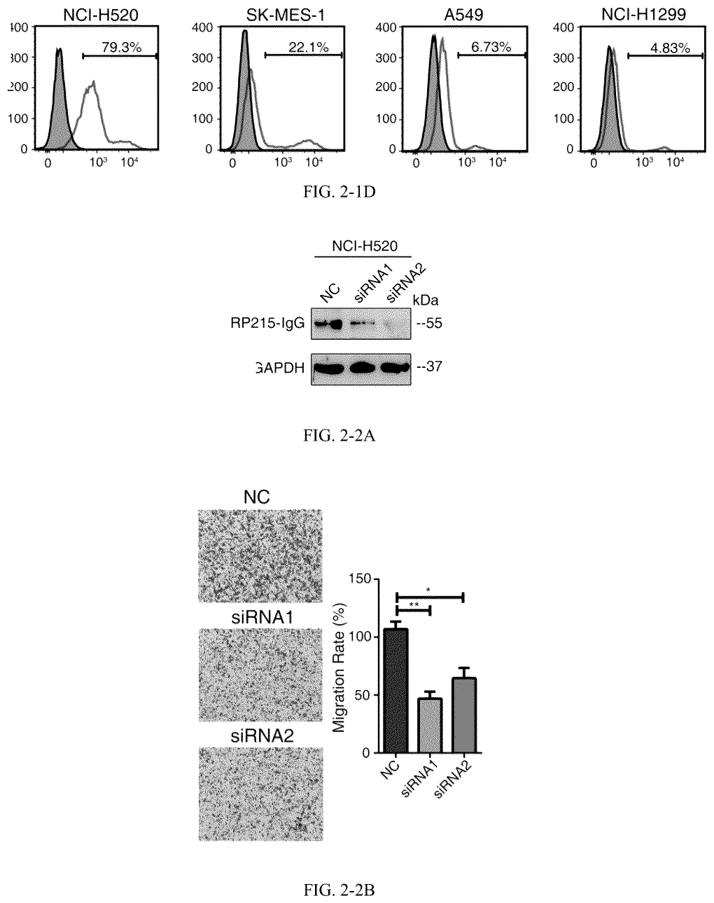

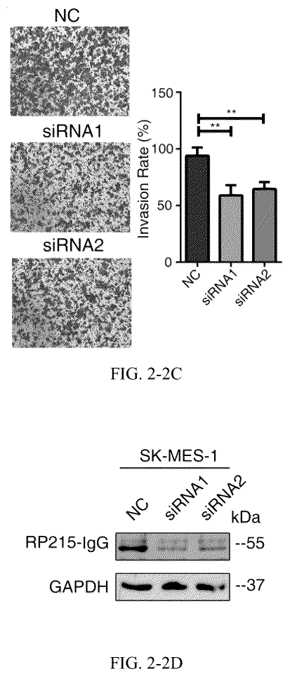

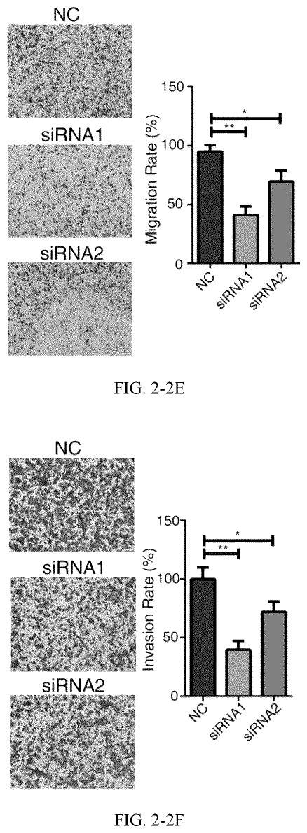

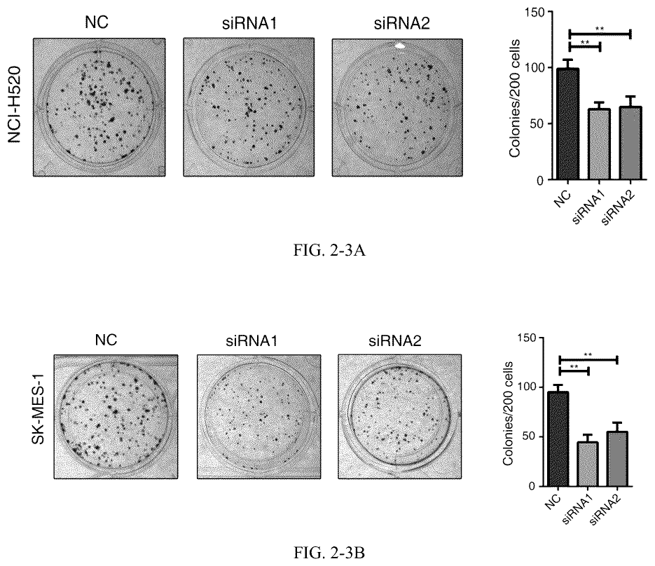

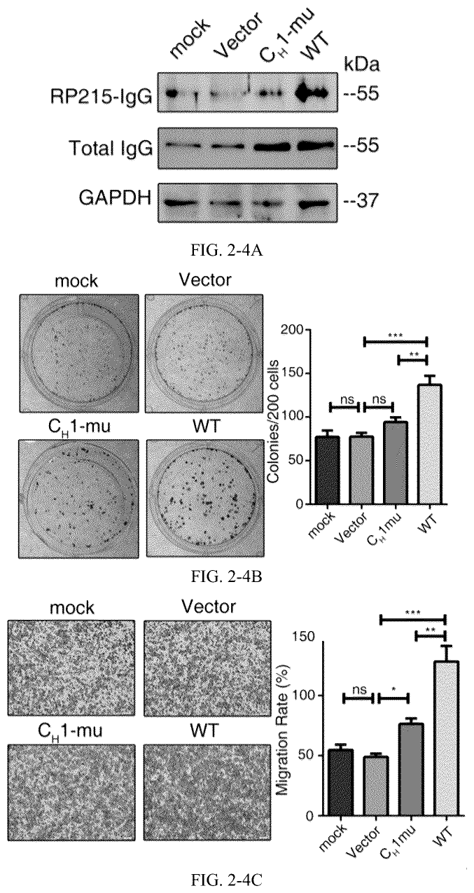

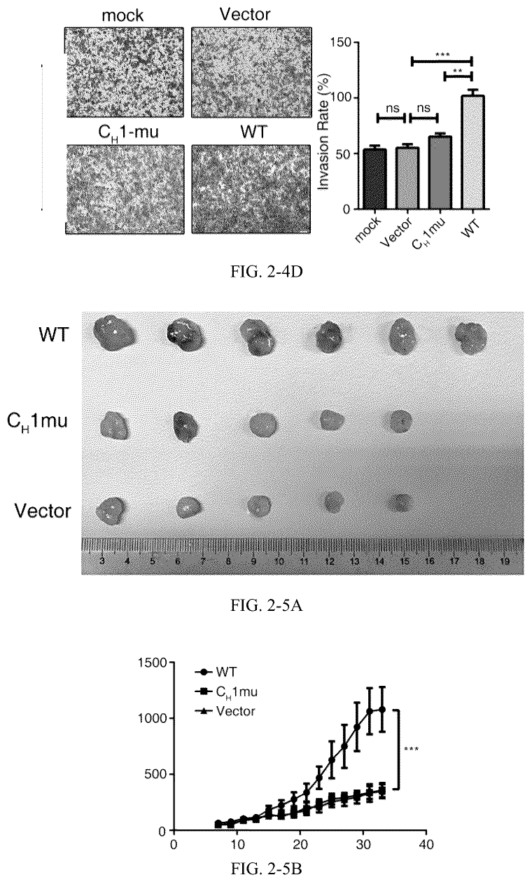

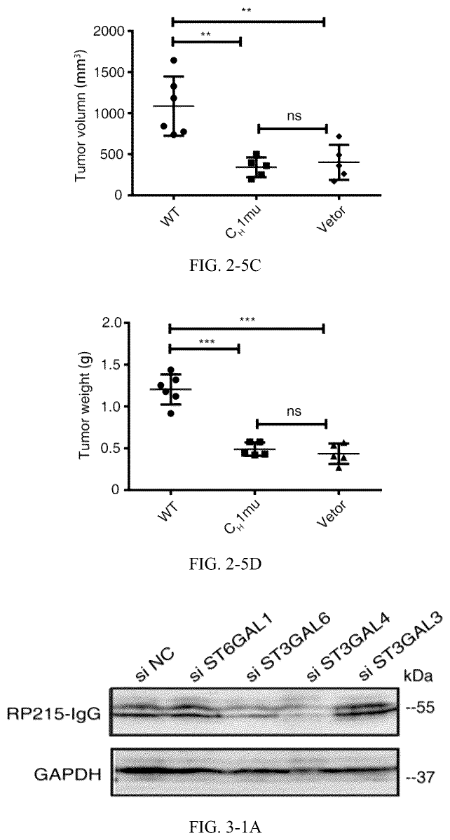

The mutation sites were shown in FIG. 1-2A, of which, WT represented wild type; C_{H}1mu represented mutation of site 162: S160A and N162C; C_{H}2mu represented mutation of site 297: N 297 Q.

Two constant regions with mutation sites were fused with variable regions V_{H}5-51/D3-9/J_{H}4 (predominant expression sequences detected in lung squamous carcinoma cells), named C_{H}1mu and C_{H}2mu, respectively, at the same time, the wild type IgG (WT) with C_{H}1 and C_{H}2 domains was constructed as a control. Firstly, these recombinant IgG plasmids (WT, C_{H}1mu and C_{H}2mu) were over-expressed in 293T cells. Western blot was used to detect the recognition of wild-type and mutant IgG by RP215. Results showed that RP215 could recognize WT and C_{H}2mu, but could not recognize C_{H}1mu (FIG. 1-2B).

Next, we verified the epitope recognized by RP215 in the lung squamous carcinoma cell line NCI-H520, thereby eliminating the interference of endogenous RP215-IgG. We constructed a recombinant IgG plasmid with a flag tag, and then used anti-flag beads for IP to obtain exogenously expressing wild-type or mutant IgG. Similar to 293T results, RP215 could recognize WT or C_{H}2mu in NCI-H520 cells very well, but it could slightly recognize C_{H}1mu (see FIG. 1-2C).

The epitope recognized by RP215 on RP215-IgG was located in the non-classical N-glycosylation site Asn162 of the C_{H}1 domain, not the classic N-glycosylation site Asn297.

Therefore, the C_{H}1 domain with N-glycosylated sialic acid modification at Asn162 site could be used as a unique epitope for non-B cell-derived IgG.

### Example 2 Identification of Non-B Cell-Derived IgG that has the Ability to Promote the Proliferation, Migration and Invasion of Tumor Cells Using IgG Epitope as a Target

Example 1 confirmed the unique epitope of non-B cell-derived IgG, which could be specifically recognized by RP215, so using this epitope as a specific recognition site, RP215 was used to detect the functions of non-B cell-derived IgG (RP215-IgG).

NSCLC cell lines: A549 (human alveolar basal epithelial cells of adenocarcinoma), Calu-3 (human lung adenocarcinoma), NCI-H1299 (human lung adenocarcinoma), NCI-H520 (human lung squamous carcinoma cells), SK-MES-1 (human lung squamous carcinoma cells).

Firstly, we detected expression of RP215-IgG in the above several NSCLC cell lines, and found that RP215-IgG that could be expressed and secreted by NSCLC cell line was localized on the cell surface and ECM (shown in FIG. 2-1A-D).

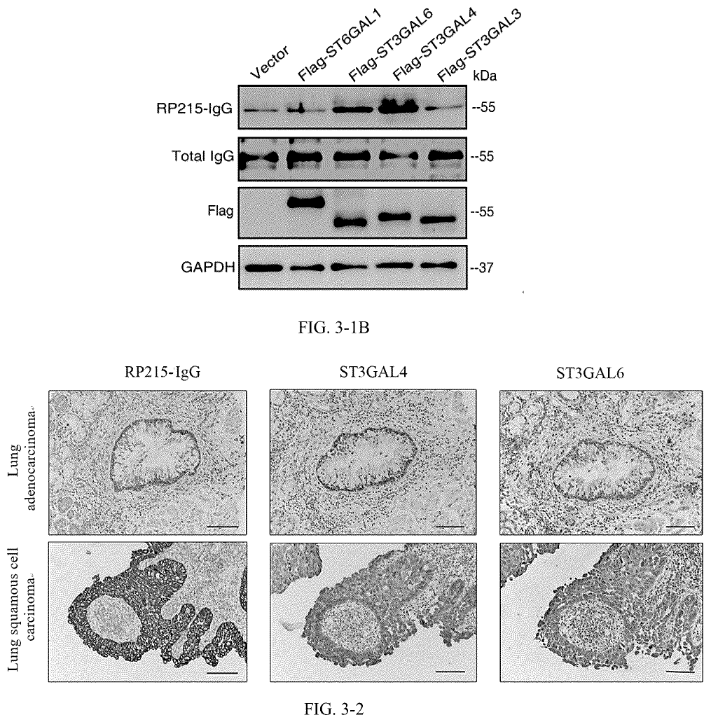

When siRNAs targeting the heavy chain constant region in NCI-H520 cells and SK-MES-1 cells downregulated IgG, the size of clones formed by these cells was reduced, and the number of clones formed was also significantly reduced. In addition, in the Transwell and Matrigel-coated Transwell assays, the migration and invasion ability of cells was significantly reduced (FIG. 2-2A-F, FIG. 2-3A-B). In addition, similar experiments indicated that sialylated IgG was associated with cell migration, invasion and metastasis in lung ADC, breast cancer and kidney cancer.

In vitro experiments, the over-expression of wild-type IgG (WT) could apparently promote cell migration, invasion, and clone forming ability. After mutating the glycosylation site of C_{H}1 domain, compared with WT, CHI mutant IgG had a significantly reduced ability to promote cell migration, invasion and colony formation, and its activity had migration-promoting effect compared to the empty vector group, but there was no significant difference in invasion ability between them (FIG. 2-4A-D).

Further, we established subcutaneous tumorigenic models of NCI-H520 cells stably expressing wild-type IgG, C_{H}1 mutant IgG and empty vector in nude mice, to observe the tumor growth promoting ability of the wild-type IgG and C_{H}1 mutant IgG. Results showed that the over-expression of wild-type IgG could significantly promote the growth of tumors, while the cells grew slowly in the C_{H}1mu group, and its number, volume and weight of tumors formed were all significantly lower than those in the WT group, but were not significantly different from those in the empty vector group (FIG. 2-5A).

In summary, the in vivo and in vitro experiments have showed that RP215-IgG can promote the survival, migration and invasion of lung squamous cell carcinoma, and its biological activity depends on the non-classical glycosylation site of C_{H}1 domain.

### Example 3 Sialyltransferase ST3GAL4 Involved in Sialylation of RP215-IgG

The biosynthesis of sialylation oligosaccharide sequences is catalyzed by a family of enzymes called sialyltransferase, and each sialyltransferase has its specific substrate.

Previous studies showed that the sialic acid linked to the N-glycan at the classical N-glycosylation site (Asn297) was mediated by sialyltransferase ST6GAL-1, and the sialic acid was connected with N-glycan β-D-galactopyranosyl (Gal) residue by α 2,6-.

We have determined that the sialic acid linked to the N-glycan at the non-classical N-glycosylation site (Asn162) was connected to the β-D-galactopyranosyl (Gal) residue via MALI, therefore, the three sialyltransferases (ST3GAL3, ST3GAL4, ST3GAL6, involved in ST3β-galactoside α-2,3-linkage) and ST6GAL1 (as a control) were the candidates for further screening.

In order to determine which sialyltransferase was involved in synthesis of RP215-IgG, RP215 was used in the Western blot of four sialyltransferase silencing. Results showed that, both ST3GAL4 and ST3GAL6 knockdown reduced the expression of RP215-IgG, and the over-expression of ST3GAL4 and ST3GAL6 resulted in an increase in the IgG recognized by RP215. It should be noted that, there was no change in Western blot of the commercial anti-IgG antibody, indicating that the IgG recognized by RP215 was greatly affected by sialic acid (Example 3-1).

In order to identify the relationship between sialylated IgG (i.e. RP215-IgG) and ST3GAL4/ST3GAL6, we analyzed the expression profile and distribution in NSCLC. Immunohistochemical results showed RP215-IgG and ST3GAL4 staining in bronchial epithelial basal cells in lung adenocarcinoma tissues and lung squamous cell carcinoma tissues, while the positive staining of ST3GAL6 was not restricted. IHC results showed that ST3GAL4 was more related to the sialylation of RP215-IgG (Example 3-2).

Therefore, blocking the activity of the ST3GAL4 enzyme can prevent the sialylation of IgG and thereby inhibit its function in tumor cell migration and invasion, and prevent the growth, migration and invasion of tumor cells.

### Example 4 RP215-IgG Interacted with Integrin α6β4 and Cross-Linked with c-Met

In order to explore the mechanism by which RP215-IgG promotes the proliferation, migration and invasion of lung squamous cell carcinoma, we first searched the RP215-IgG interacting protein. The lung squamous carcinoma cell line NCI-H520 protein was extracted, and RP215 was used for immunoprecipitation (immunoprecipitation, IP), then all proteins obtained by the antibody RP215 and the control antibody mIgG IP were analyzed by LC-MS/MS.

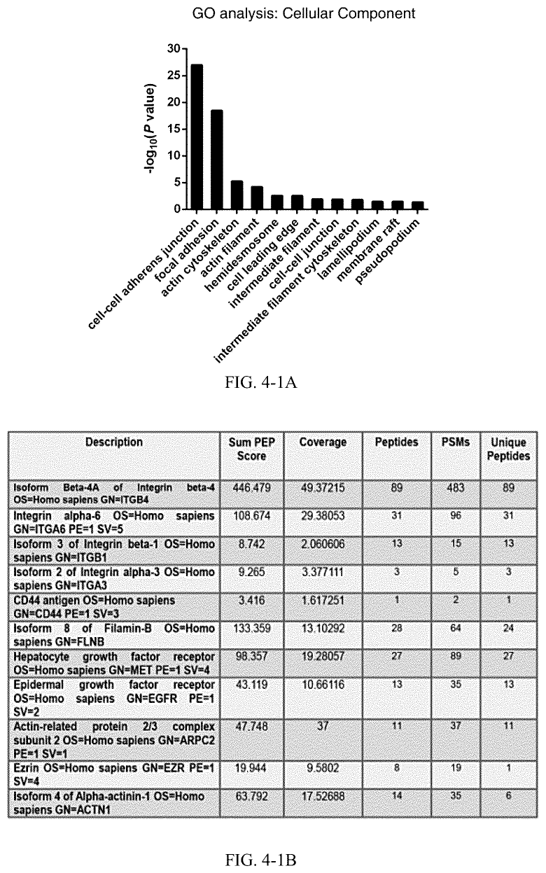

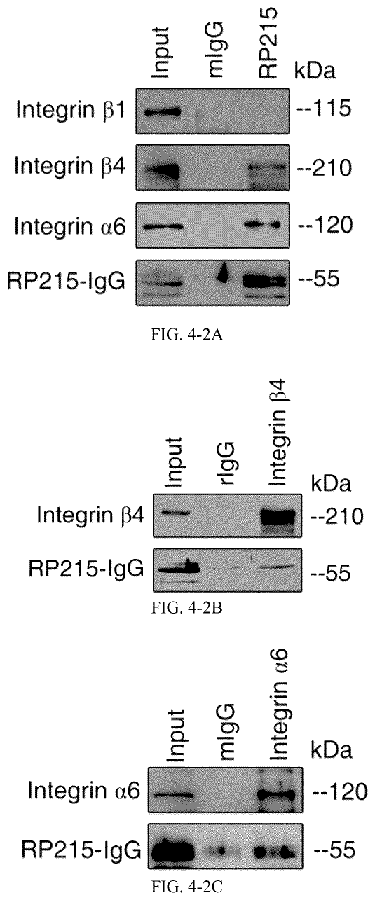

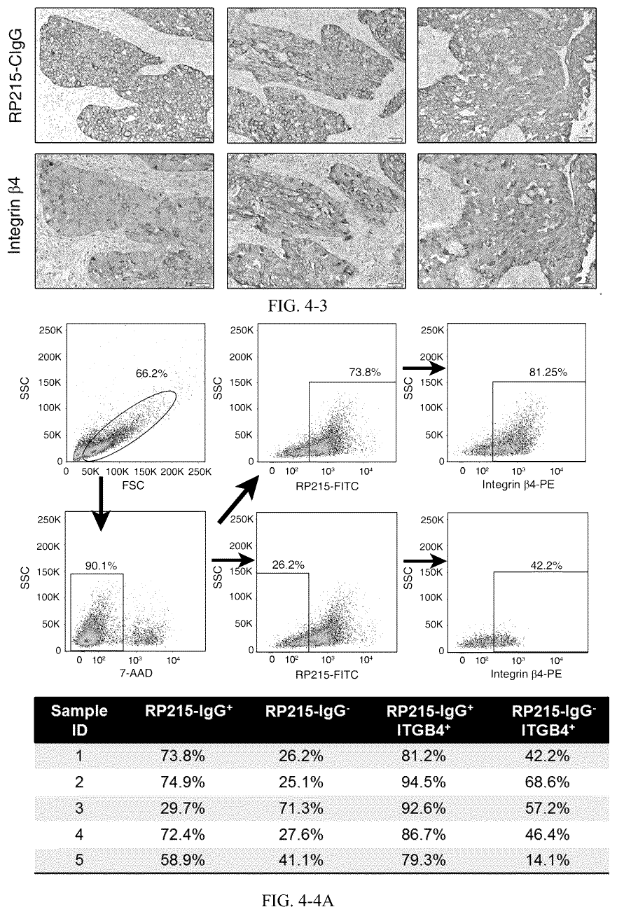

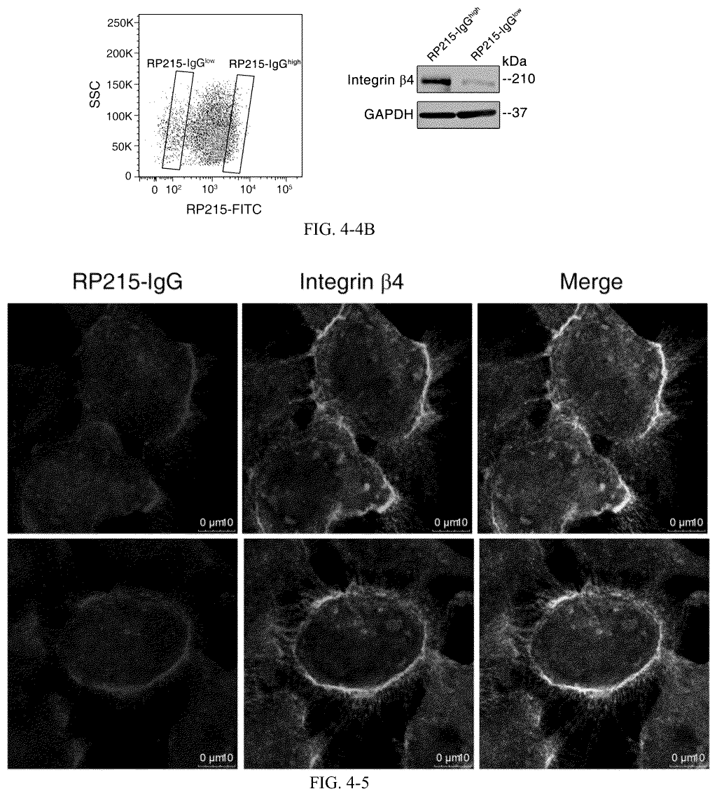

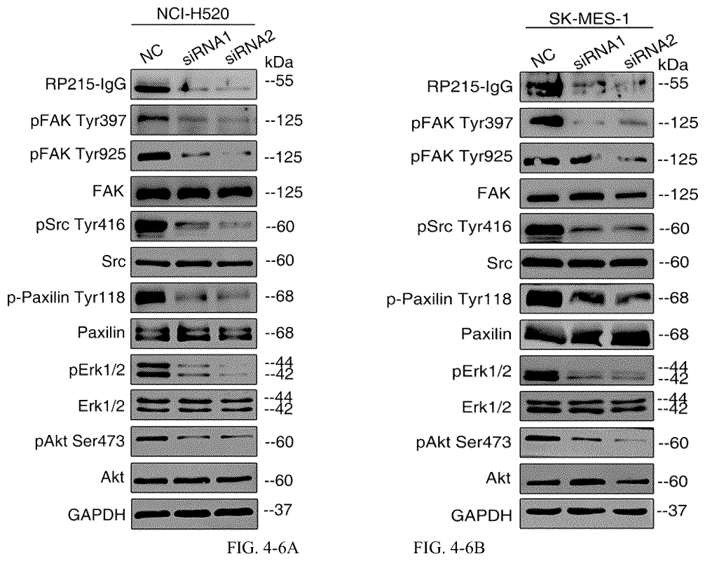

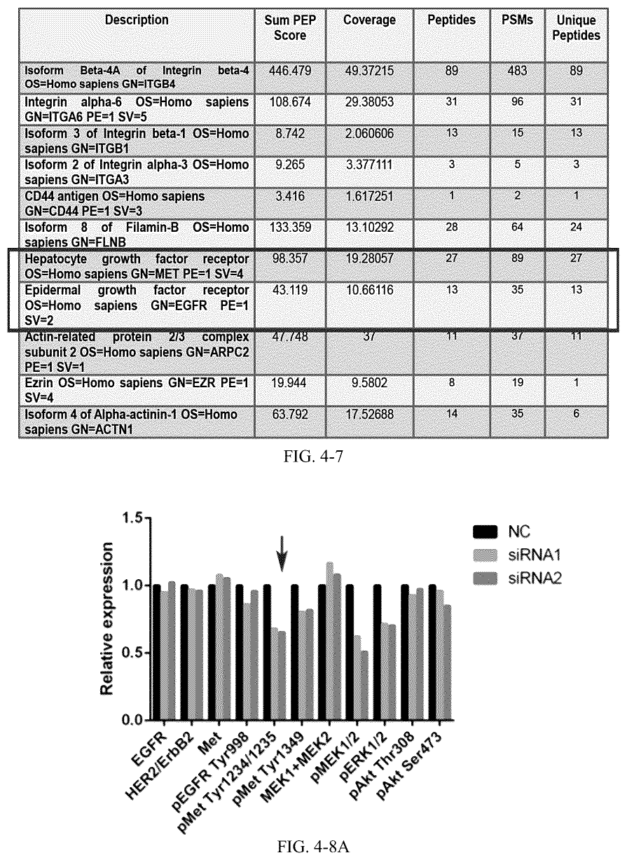

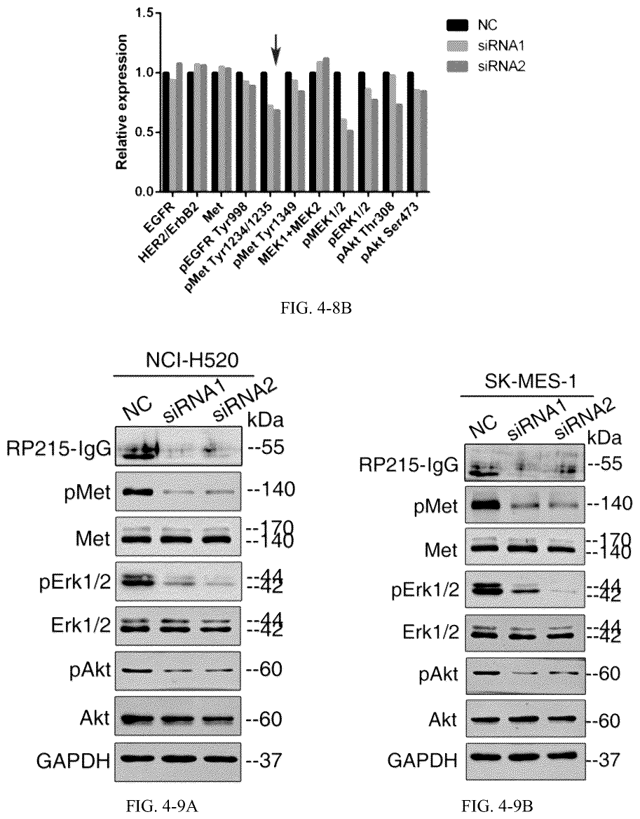

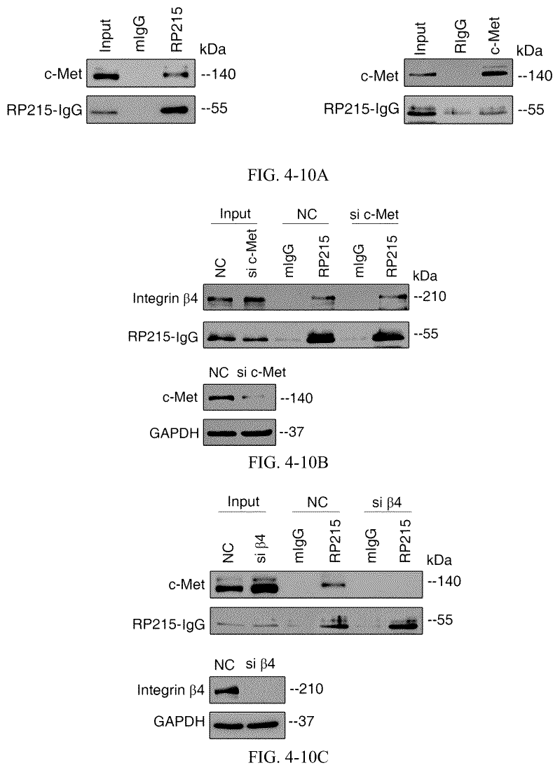

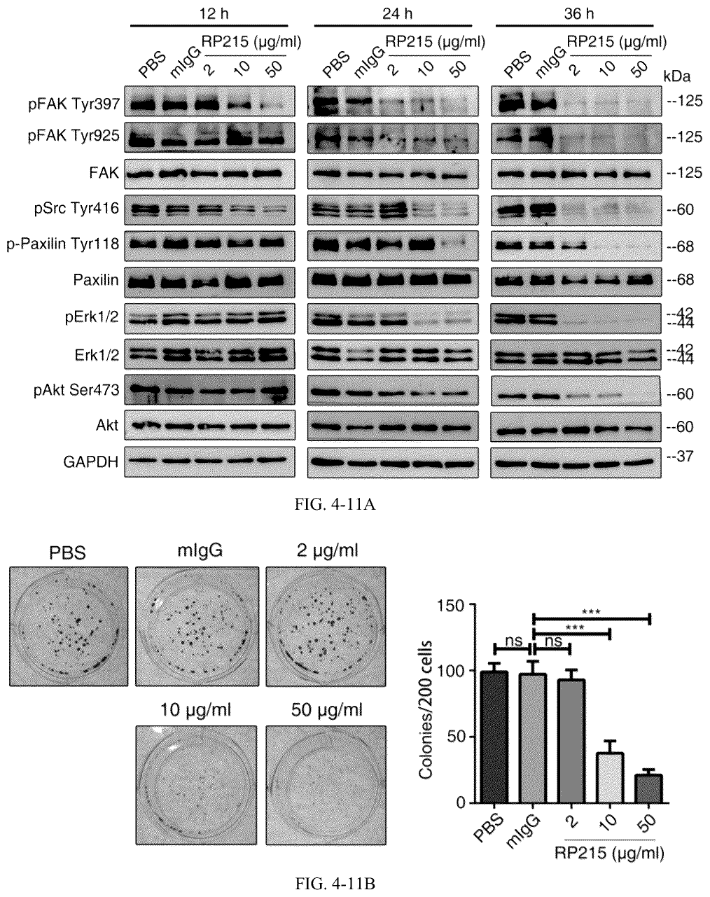

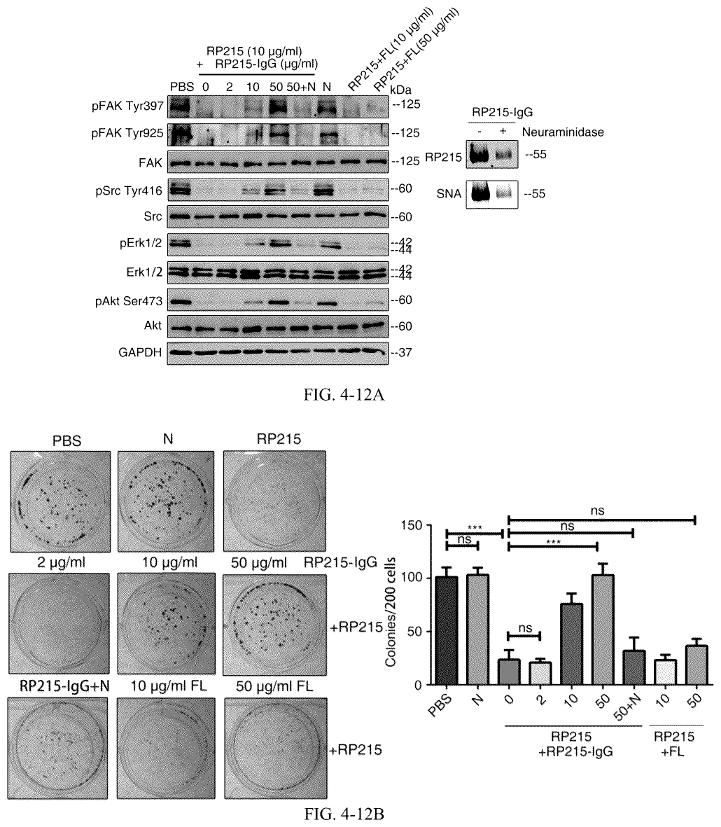

While the potentially interacting proteins were analyzed by MS, we quantified the relative abundance of these proteins by label-free quantification (LFQ) method. The protein obtained with RP215 IP was compared with the protein obtained with the negative control antibody mIgG IP, and we concluded that only the proteins with an LFQ value of zero in the mIgG group were proteins that could specifically interact with RP215-CIgG. Next, Gene Ontology (GO) analysis was conducted for all proteins screened from database david.ncifcrf.gov, that is, Gene Ontology Cellular Component analysis. Results showed that the protein components that specifically interacted with RP215-IgG were mainly cell membrane-related proteins, which were involved in the cell-cell adhesion junctions, focal adhesions, and formation of hemi-desmosomes (FIG. 4-1A). We further analyzed the protein components in these GO terms and compared their MS scores (results were shown in FIG. 4-1B), finally we chose to focus on integrin family, which played an important role in promoting tumorigenesis and metastasis based on several reports in the recent years.

In order to confirm our MS findings, we performed IP (immunoprecipitation) with RP215 and found that sialylated IgG (i.e. RP215-IgG) interacted with integrin β4 or integrin α6, but had no interaction with integrin β1. At the same time, immunoprecipitation was performed with integerr β4 or integerr α6, and RP215-IgG was detected in their affinity elution fractions, indicating the specific interaction between sialylated IgG and integrin α6β4 complex (FIG. 4-2A-C).

In order to clarify whether the expression of sialylated IgG is related to the expression of integrin β4, we first used clinical lung squamous cell carcinoma tissues to detect the tissue distribution pattern of sialylated IgG and integrin β4 on adjacent paraffin sections by immunohistochemistry. We found that both RP215 and anti-integrin β4 antibody had strong positive staining on the cell membrane in lung squamous cell carcinoma, and their distribution patterns were very similar (FIG. 4-3).

In addition, we explored the correlation between RP215-IgG and integrin β4 expression levels at the cellular level. We digested the tumor tissue of the lung squamous cell carcinoma PDX model with collagenase IV and DNase I to obtain a single cell suspension, and then analyzed their expressions on the cell membrane by flow cytometry. After viable cells were obtained with 7-AAD negative gates, RP215-IgG was highly expressed in PDX tumors. The further flow cytometry showed that the positive rate of integrin β4 in RP215-IgG positive cell population (79.3%-92.6%) was significantly higher than the positive rate of integrin β4 in RP215-IgG negative cell population (14.1%-57.2%) (FIG. 4-4A).

In addition, we evaluated the expression of integrin β4 in RP215-IgG positive cells in lung squamous carcinoma cell lines. We used flow cytometry sorting to obtain two groups of cells with high expression of sialylated IgG (RP215-IgGhigh) and low expression of sialylated IgG (RP215-IgGlow) by enrichment from NCI-H520 cell line, then the expression levels of integrin β4 in the two groups of cells were detected by Western blots. Results showed that RP215-IgG was positively correlated with integrin β4 protein expression level in LSCC cell line (FIG. 4-4B).

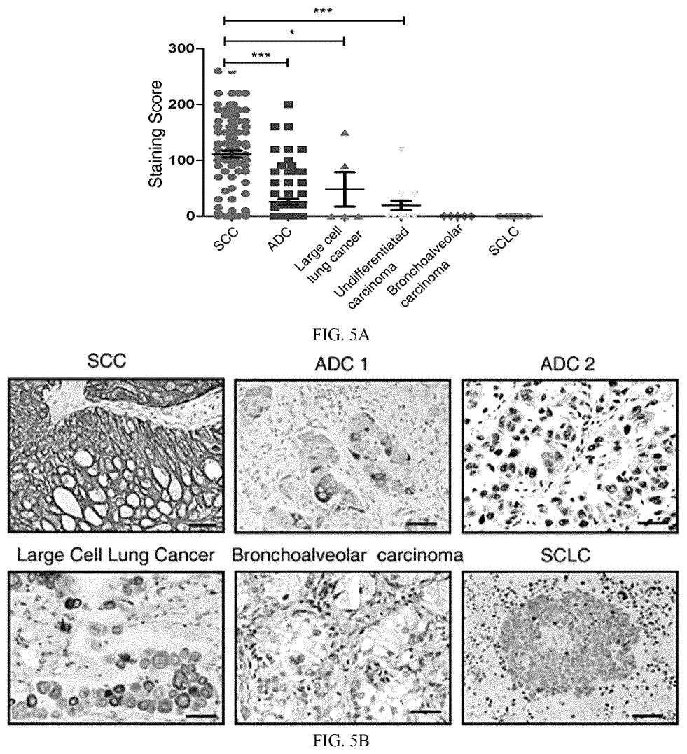

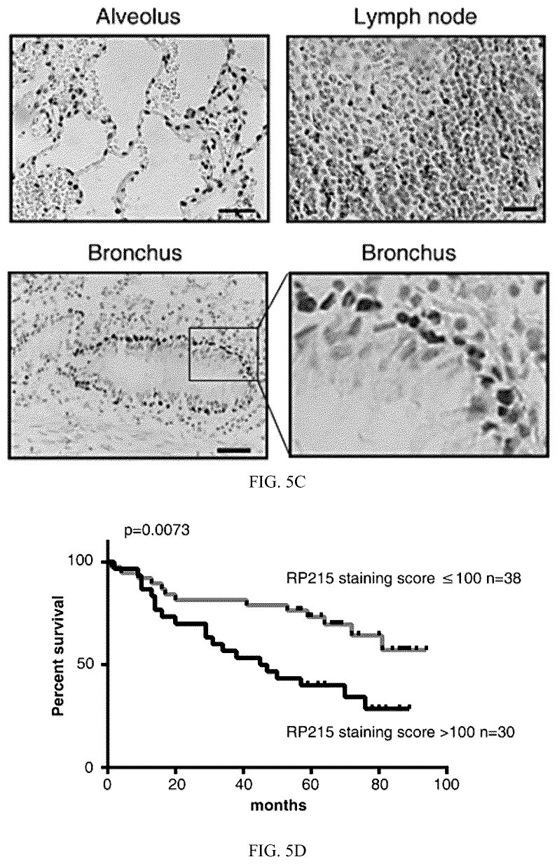

Finally, we detected the localization of RP215-IgG and integrin β4 in cells using immunofluorescence method. The results showed that, RP215-IgG was expressed in the cell membrane and cytoplasm, integrin β4 was mainly expressed on the cell membrane, and they had obvious co-localization on the cell membrane (FIG. 4-5).

In summary, we found that RP215-IgG and integrin β4 were co-expressed and co-localized at the tissue and cellular levels, which provided a basis for the interaction between them under natural conditions.

The co-expression and co-localization of RP215-IgG and integrin β4 also proved that integrin β4 could be used as a marker to characterize the distribution and expression of RP215-IgG, which could be used for the detection of RP215-IgG. Since RP215-IgG could promote the proliferation, migration and invasion of epithelial tumors, integrin β4 could be used as a marker for preparing drugs for the auxiliary detection of epithelial tumors.

In malignant tumor cells, integrin α6β4 has been shown to be associated with multiple receptor tyrosine kinases (RTK), which can amplify the signals to promote the invasion and metastasis of tumor cells. We used antibody RP215 to perform immunoprecipitation in NCI-H520 cells, and used mass spectrometry to find RP215-IgG interacting proteins. Results showed that c-Met and EGFR of the RTK family were also detected in the components bound to RP215, and their scores were relatively high, suggesting that interaction exists between RP215-IgG and the RTK family (FIG. 4-7).

In order to study the specific RTK activated by sialylated IgG, we knocked down IgG in NCI-H520 and SK-MES-1, and detected EGFR, HER2, c-Met in the RTK family and the level of phosphorylation of these key molecules related to RTK downstream signal transduction by phosphorylation chips. Results showed that, after knockdown of IgG in two cell lines, the phosphorylation level at the site Tyr1234/1235 of c-Met was significantly down-regulated, while the phosphorylation levels of EGFR and HER2 showed no significant change (FIG. 4-8A-B). At the same time, the phosphorylation levels of Ras-MAPK at the downstream of RTK and key molecules MEK, Erk1/2, and Akt of PI3K-Akt pathway were decreased significantly. We further confirmed the results of phosphorylation chips using Western blots (FIG. 4-9A-B).

Since IgG knockdown reduced Met tyrosine phosphorylation greatly, we explored how sialylated IgG formed complexes with integrin β4 or Met in LSCC.

First, we confirmed the interaction between RP215-IgG and c-Met in NCI-H520 cells by endogenous co-immunoprecipitation. Subsequently, we explored the interactions between RP215-IgG, integrin α6β4, and c-Met by co-immunoprecipitation method. We knocked down c-Met and integrin β4 in NCI-H520 cells respectively, and then used RP215 for immunoprecipitation. Results showed that, compared with the non-knockdown group NC, the knockdown of c-Met did not affect the interaction between RP215-IgG and integrin β4, but after knockdown of integrin β4, the interaction between RP215-IgG and c-Met disappeared (FIG. 4-10B, C). In conclusion, it suggested that sialylated IgG and integrin β4 must form a complex before interacting with Met.

The integrin-FAK and Met signaling pathways were involved in the cell proliferation and migration regulated by sialylated IgG.

Subsequently, we studied the molecular mechanism of sialylated IgG to promote the proliferation and migration of LSCC cell lines. The integrin family, as extracellular matrix protein receptor, has no intrinsic tyrosine kinase activity, but mainly performs signal transduction by recruiting and activating non-receptor tyrosine kinases. When binding to the corresponding ligand, integrin β4 can recruit focal adhesion kinase (FAK) through the domain of its cytoplasmic region, to phosphorylate the Tyr397 site of FAK, then bind to the SH2 domain of Src and promote the phosphorylation of Tyr416 site of Src; after phosphorylation of Src, the Tyr925 site of FAK can be phosphorylated through feedback regulation, which eventually leads to downstream Ras-MAPK or PI3K-Akt cascade reaction.

In order to identify whether sialylated IgG was involved in FAK or Met signaling, IgG was silenced in LSCC cell lines by siRNA. Apparently, after knocking down IgG with two different siRNAs, the phosphorylation levels of FAK and Src-related sites could be significantly down-regulated, indicating that the down-regulation of RP215-IgG expression level could lead to inactivation of FAK-Src signal transduction. In addition, as a member of the focal adhesion complex, paxilin is a direct activation target of FAK, and the up-regulation of phosphorylation level at the site Tyr118 can activate paxilin and function as a cytoskeletal adaptor protein, thereby activating cell movement or cell polarization-related signal paths. We also found that when IgG was knocked down, the phosphorylation level of paxillin at the site Tyr118 was also significantly inhibited (shown in FIG. 4-6A-B).

Because sialylated IgG could be secreted into the supernatant and co-localized with integrin 34 on the LSCC cell membrane, we used monoclonal antibody RP215 to block and destroy the complex formed by IgG and integrin β4, to investigate whether the observed changes in FAK signal in IgG-silenced LSCC cells were induced by secreted IgG. Compared with the isotype control, after treatment with RP215, the phosphorylation of FAK, Src, paxilin and Akt was significantly reduced in a concentration- and time-dependent manner, and the activation of Erk1/2 mediated by Met signal transduction was greatly reduced (experimental results were shown in FIG. 4-10A-C).

In order to further verify the effects of secretory IgG, exogenous sialylated IgG (RP215-IgG) purified from LSCC PDX tumors by RP215 affinity chromatography was added to the culture medium of NCI-H520 cells to rescue FAK signal transduction caused by exogenous addition of RP215 or knockdown of IgG. It was found that by incubating with RP215-IgG at an increased dose, the inhibition of FAK signal transduction was reversed gradually. In addition, exogenous addition of RP215-IgG could significantly rescue the decrease in clonal formation and migration ability caused by IgG knockdown, but the IgG components of flow-through liquid in RP215 affinity chromatography could not achieve this function (FIGS. 4-11A-B and FIGS. 4-12A-B).

In order to further study whether the activation of FAK signal transduction neutralized by sialylated IgG depended on its N-glycan modification of sialylation, RP215-IgG was digested with neuraminidase. When incubated with 10 μg/ml of RP215 for 36 hours, we did not observe the effect of RP215-IgG on FAK activity after digestion with neuraminidase, indicating that the functional activity of sialylated IgG depended on its sialic acid structure.

To sum up, integrin-FAK signal transduction is the key molecular mechanism of sialylated IgG to promote the proliferation and migration of cancer cells.

Previous studies have shown that sialylated IgG bound to integrin α6β4 to form a complex, to promote the activation of integrin-FAK signaling pathway. Therefore, sialylated IgG can be used as a ligand of integrin α6β4 for preparing drugs for the diagnosis or treatment of diseases mediated by 0604-FAK pathway. Of course, the Asn162 site of C_{H}1 domain of the IgG is modified by N-glycosylated sialic acid.

### Example 5 Specific Labeling of Non-Small Cell Lung Cancer with RP215-IgG

### Patient Samples:

Formalin-fixed, paraffin-embedded lung cancer tissue sections were obtained from 242 patients in Harbin Medical University Cancer Hospital (Harbin, Heilongjiang Province). The clinicopathological features were available from the review of medical records. The diagnosis and histological classification of tumor specimens were based on WHO classification. The stage of tumor-lymph node metastasis (TNM) was determined according to the guidelines of the American Joint Committee on Cancer (AJCC).

All patients were 25 to 82 (56.6±10.6) years old, including 121 cases of SCC (squamous cell carcinoma), 76 cases of ADC (lung adenocarcinoma), 21 cases of SCLC (small cell lung cancer), 5 cases of large cell lung cancer, 5 cases of bronchoalveolar carcinoma, and 14 cases of undifferentiated carcinoma. 62 patients (25.6%) were women. In terms of histopathological grading, 26.8% of samples (65 cases) were well differentiated (grade I), 49.2% (119 cases) were moderately differentiated (grade II), and 24.0% (58 cases) were poorly differentiated (grade 3). According to the TNM staging criteria, 134 patients (55.4%) were in stage I, 50 patients (20.7%) were in stage II, 56 patients (23.1%) were in stage III, and 2 patients (0.8%) were in stage IV.

The sialylated IgG was determined using the monoclonal antibody RP215 in 242 cancer tissues of patients with different types of lung cancer.

We first found a high expression of sialylated IgG in NSCLC (140/221, 63%), but not found in SCLC (0/21). In addition, sialylated IgG was expressed at high frequency in SCC (102/121, 84.3%) in NSCLC cases; while expressed at low frequency in ADC ( 28/76, 36.8%), small cell lung cancer (⅖, 40.0%) and undifferentiated cancer ( 8/14, 57.1%); no staining was observed in bronchoalveolar carcinoma (0/5).

We compared the expression pattern and pathological score of sialylated IgG, and found that all cancer cells, especially the cell surface, showed very strong staining (score: 110.9) in sialylated IgG positive tissues of SCC. However, only a few cancer cells showed weak or moderate staining in non-SCC tissues (score: 21.1), indicating that the sialylated IgG could also promote the progression of NSCLC, and sialylated IgG on the cell surface of SCC could be used as a target for lung SCC therapy.

Subsequently, we explored whether sialylated IgG could be expressed in normal lung tissues. When draining lymph nodes from autopsy (6 cases) or adjacent cancer tissues (23 cases), we found that pseudostratified columnar ciliary epithelial cells, normal alveolar epithelial cells and lymphocytes of draining lymph node were not stained. However, peripheral staining was observed in basal cells of bronchial epithelial cells. So far, LSCC is considered to be derived from this cell population, especially the bronchial hyperplasia basal cells adjacent to the cancer tissue (FIG. 5A-C).

### Example 6 Specific Labeling of Other Epithelial Tumors with RP215-IgG

### Patient Samples:

Cancer tissue sections included 100 patients with colorectal cancer, 200 patients with breast cancer, 87 patients with prostate cancer, 70 patients with kidney cancer, 45 patients with bladder cancer, 80 patients with salivary gland cystadenocarcinoma, 70 patients with gastric cancer, 20 patients with pancreatic cancer and 50 patients with esophageal cancer; and they were purchased from Shaanxi Chaoying Biotechnology Co., Ltd. and Shanghai Outdo Biotech Co., Ltd. The clinicopathological features were available from the review of medical records. The diagnosis and histological classification of tumor specimens were based on WHO classification. The stage of tumor-lymph node metastasis (TNM) was determined according to the guidelines of the American Joint Committee on Cancer (AJCC).

RP215-IgG was determined in different types of epithelial tumors using monoclonal antibody RP215.

Results showed that the expression frequencies were varied in different cancer tissues: 74% ( 74/100) in colorectal cancer, 94.5% (189/200) in breast cancer, 85% ( 74/87) in prostate cancer, 77% ( 54/70) in kidney cancer, 100% ( 54/54) in bladder cancer, 94% ( 75/80) in salivary gland cystadenocarcinoma, 86% ( 60/70) in gastric cancer, 100% (20/20) in pancreatic cancer and 100% (50/50) in esophageal cancer. Apparently, RP215-IgG was highly expressed in a variety of tumors of epithelial origins.

When draining lymph nodes from autopsy (6 cases) or adjacent cancer tissues (23 cases), we found that pseudostratified columnar ciliary epithelial cells, normal alveolar epithelial cells (left upper corner of FIG. 5C) and lymphocytes of draining lymph node (right upper corner of FIG. 5C) were not stained. However, peripheral staining was observed in basal cells of bronchial epithelial cells (two figures below FIG. 5C). So far, LSCC is considered to be derived from this cell population, especially the bronchial hyperplasia basal cells adjacent to the cancer tissues.

We compared the relationship between expression frequency and tumor metastasis and poor prognosis, and found that the expression level and frequency of RP215-IgG were positively correlated with tumor metastasis and poor prognosis. This suggested that RP215-IgG is involved in the occurrence and metastasis of the above epithelial tumors, with a variety of therapeutic targets for the epithelial tumors, and can be used to predict tumor metastasis and poor prognosis (FIG. 5 D).

### Example 7 Treatment of Cancers by Blocking RP215-IgG

### Functional Blocking Antibody RP215 Showed Therapeutic Effect in LSCC PDX Models In Vivo

The previous experimental results confirmed that the N-glycosyl group at the site Asn162 of C_{H}1 domain of RP215-IgG could be identified by RP215 after modified by sialic acid, and the glycosylation modification of IgG with this functional structure was mediated by sialyltransferase ST3GAL4. The monoclonal antibody RP215 with unique structure was identified to bind RP215-IgG to block its function.

It was involved in the migration and invasion of tumor cells.

Establishment of PDX tumor model and antibody therapy:

The Beige mice with severe combined immunodeficiency (SCID) were obtained from Vital River Laboratories Technology Co., Ltd. (Beijing, China) at 6 weeks. The animal care and uses were carried out according to the guidelines for animal medication and nursing of the Peking University Health Science Center.

The tumor tissues of 3 patients were obtained from those with lung squamous cell carcinoma who underwent surgical resection at Peking University Cancer Hospital. The fourth generation of PDX was used for antibody therapy. The metastatic tumor was placed in a sterile Petri dish containing RPMI 1640, and then cut into tissue blocks with size of 2×2×2 mm³. Typically, each segment was implanted into the right and left subcutaneous areas. Antibody therapy was started after the tumor reached around 100 mm³. The mice were randomly assigned to their respective treatment groups. The mIgG or RP215 (dissolved in PBS) was injected via the tail vein at a rate of 5 mg/kg, twice a week, for 6 weeks. The tumor growth condition was monitored every other day.

After understanding the carcinogenic properties of RP215-IgG in LSCC, we detected whether RP215 could establish the therapeutic effects of the PDX models by intravenous injection.

PDX tumors retained most of the key genes expressed in the primary tumor and were closer to the original clinical cancer than the originally established cell line. In our study, we used the tumors of 3 patients with LSCC and the histological analysis of transplanted tumors confirmed that xenografts maintained the LSCC phenotype.

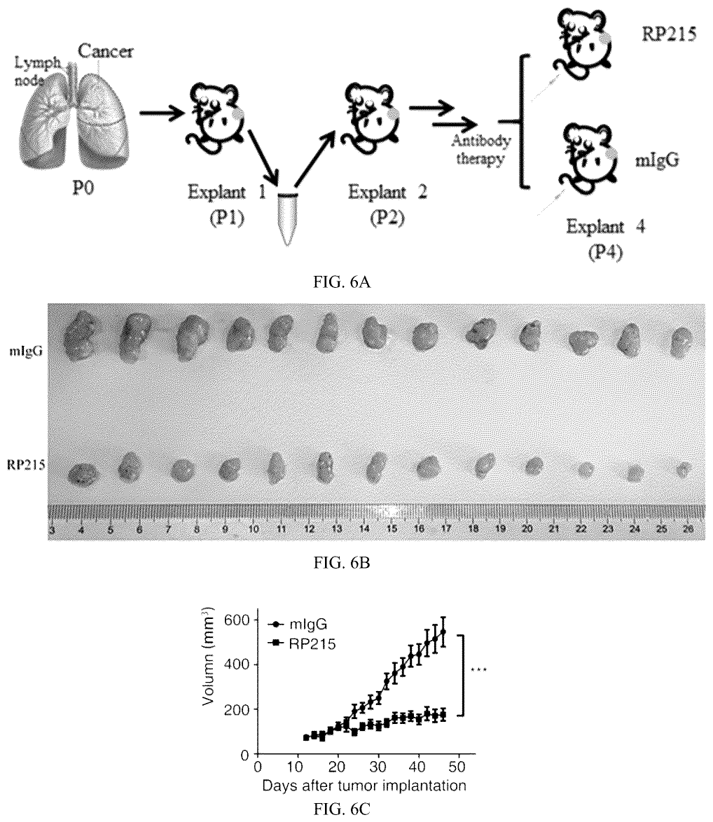

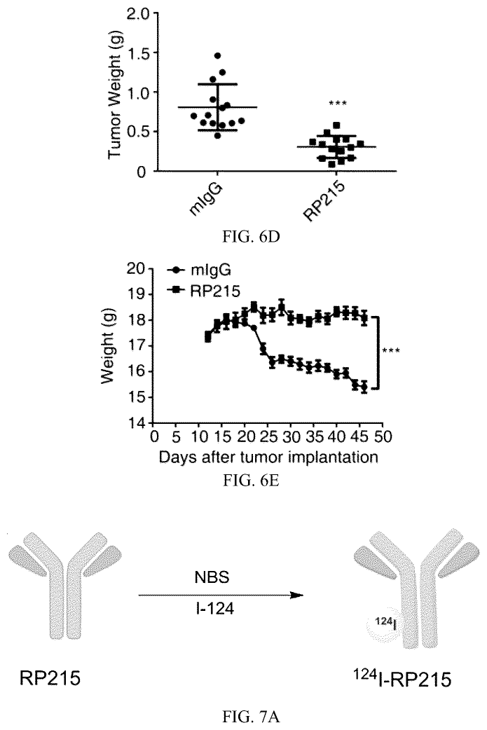

As shown in FIG. 6A-E, compared to the mIgG control, RP215 at a dose of 5 mg/kg per mouse reduced the growth of pre-established tumor by 60%. At the end of the experiment, the average weight of tumors in the RP215 treatment group was 0.31±0.14 g, significantly lower than that in the control group (0.81±0.29 g). Similar effects were observed in other two conditions.

### Example 8 Production, Quality Control and Preliminary Micro-PET/CT Study of ¹²⁴I-RP215

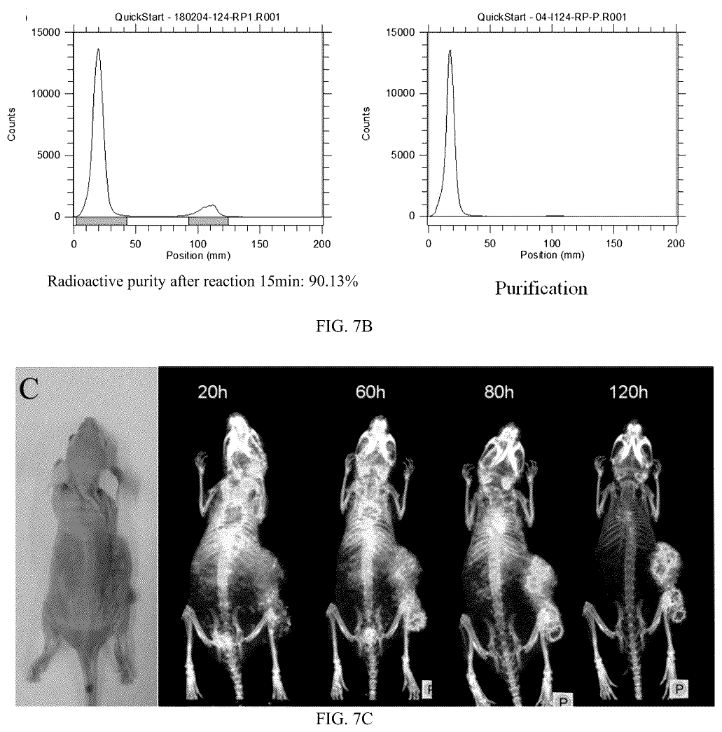

¹²⁴TeO₂ (99.0%) was purchased from Center of Molecular Research of Russia, Sumitomo HM-20 cyclotron and ¹²⁴I purification system (Industrial Equipment Division) were purchased from Sumitomo Corporation of Japan, radioactivity activity meter was purchased from U.S. Capintec, and Micro-PET/CT was purchased from Mediso Hungary. The schematic diagram of radioactive labels was shown in FIG. 7A.

### Labeling and Quality Control:

1. Labeled antibody: RP215, dosage:0.2 mg, antibody concentration: 2.0 mg/ml.
2. Radioactive ¹²⁴I: produced by the cyclotrons of the unit, with batch number of 2018-I-124-003, radioactive concentration: 5×37 MBq/mL.
3. Purification column: PD-10 column of U.S. Sigma.
4. Oxidizing agent: Bromosuccinimide (abbreviated as NBS), U.S. Sigma.
5. Phosphate buffer solution (abbreviated as PB): 0.1 and 0.01M phosphate buffer solution at pH 7.2 and pH 7.4, prepared by ourselves.
6. Human serum albumin (HSA): content of 10% (diluted with 20% HAS), produced by North China Pharmaceutical Factory.

Experimental instruments: activity meter: (National Institute of Metrology, China), surface contamination meter (Sweden), radioactive iodine-labeled glove box (Shanghai Tongpu Co., Ltd.)

### Labeling of Antibody Solution:

7. Take 0.2 mg RP215 (with volume of 200 μL), then add 0.3 ml 0.1M PB at pH 7.4 into it.
8. 0.5 ml of the required radioactive Na¹²⁴I solution was extracted, with a radioactivity of 2.5×37 MBq, added to the antibody solution to be labeled, and 10 μg of NBS (equivalent to adding 50 μg of NBS per mg of antibody, prepared by 0.01M PBS) was added immediately, after reaction for 60 s, 0.3 ml of 10% HSA was added to terminate the reaction, and then samples were taken to determine the labeling rate, purified and separated through PD-10 column. The volume of upper column: 1.5 mCi, production: 2.0 mCi, radioactivity specific activity: 10 mCi/mg; radioactivity concentration: 1 mCi/mL.
9. Determination of labeling rate: using acetone developing agent. ITLC-SG was used as a stationary phase to carry out uplinking expansion, with the marker at the origin and the free ¹²⁴I at the front. The radioactivity count was determined by Radio-TLC, and the labeling rate was calculated.

The Radio-TLC analysis of radiolabels before and after purification was shown in FIG. 7B.

### Micro-PET Imaging of ²⁴I-RP215

The ¹²⁴I-RP215 physiological saline solution of 18.5 MBq was injected into PDX model animals of human lung squamous cell carcinoma via the tail veins. At 20 h, 60 h, 80 h, and 120 h after drug injection, Matrx VIP 3000 animal anesthesia machine was used to anesthetize the mice by blowing 3.0% isoflurane with 300 mL/min of oxygen. The mice were fixed in the prone position on the scanning bed, and maintained in anesthesia state by blowing 1.0-1.5% isoflurane with 150 mL/mi oxygen, and then Micro-PET/CT scan was performed. The scanning energy window was 350-700 KeV, cross-sectional field of view was 80 mm, and the 3D mode acquisition lasted 15 minutes. After the acquisition was completed, the random and scattering attenuation correction was used to reconstruct the 3D images with the Osem algorithm. After the reconstruction, image processing was performed by software MMWKS SUPERARGUS.

After the prepared ¹²⁴I-RP215 solution was injected into normal mice via tail veins, imaging experiments was conducted by Micro-PET/CT. The results were shown in FIG. 7C. At 20 h, 60 h, 80 h, and 120 h after injection of ¹²⁴I-RP215 solution via the tail veins, the radioactive enrichment was significantly enriched from the heart blood pool to the tumor, while radioactivity concentration in other normal tissues and organs was low. In addition, the ¹²⁴I was mainly metabolized from the bladder. Micro-PET imaging demonstrated that the ¹²⁴I-RP215 prepared in this study conformed to the metabolic behaviors of iodine series monoclonal antibodies in vivo and showed very good radioactive uptake in tumors of model animals.

### Example 9. Preparation of Immunogens

The purpose of this example is to prepare different immunogens for immunizing animals or humans to generate antibodies.

The following immunogens were prepared:
1. Intact RP215-IgG (referred as “SIA-IgG” below):
(1) total protein extract from lung squamous cell carcinoma tissue: 200 mg of cancer tissue was added to 1 ml of RIPA lysis solution (150 mmol/L NaCl, 50 mmol/L Tris, 1% Triton X-100, 1% sodium deoxycholate, 0.1% SDS, 10 mM EDTA, pH 7.5, cocktail protease inhibitor), and placed on ice for 30 minutes. The sample was, centrifuged at 13000 rpm 4° C. for 20 minutes. The supernatant is the total protein. Alternatively, the cancerous ascites can be centrifuged (1000 rpm/min, 10 minutes) to obtain the supernatant.
(2) Purification of total IgG using Protein G: 2 ml of Protein G column packing was loaded in a chromatography tube, washed and equilibrated with 20 ml of PBS (pH 7.4). The total protein lysis solution was diluted with PBS and incubated overnight with Protein G column at 4° C. The column was washed with PBS to remove unbound and non-specific binding proteins. The column was eluted with 0.1 mol/L glycine hydrochloric acid buffer (pH 2.4), and the eluted IgG was neutralized with 0.1 mol/L Tris HCl (pH 11.0) to pH 7.4 in order to prevent protein denaturation. The eluted IgG was concentrated by PBS ultrafiltration and replaced into a PBS environment, and the purified IgG was aliquoted. Two ml of Protein G column packing was placed in a chromatography tube. The column was washed with approximately 20 ml PBS (pH 7.4) and equilibrated. Three ml of the preserved ascites were loaded onto Protein G column, then 6 ml of PBS was added to dilute the ascites and incubated overnight at 4° C.
(3) Purification of RP215-IgG from total IgG: 10 mg (1 mg/ml) of ascites IgG was loaded onto RP215-CNBr affinity chromatography column, for overnight at 4° C., to allow SIA-IgG to bind to the RP215-CNBr affinity chromatography column. The steps of elution and neutralization were the same as the purification steps for IgG from tumor tissue. The purified SIA-IgG was isolated and aliquoted.
2. Fab segment of SIA-IgG (referred as “SIA-IgG Fab” below): The Fab fragment of RP215-IgG is composed of C_{H}1 and the variable regions.

The ratio of papain (Sigma) to SIA-IgG is 1:20; the concentration of SIA-IgG was adjusted to 1 μg/l. The reaction solution is pH 7.6 PBS containing 10 mmol/L L-Cys and 2 mmol/L EDTA. SIA-IgG was digested at 37° C. for 4 hours before terminating the reaction with iodoacetamide (final concentration of 20 mmol/L). Fab fragments that do not bind to the Protein G column were collected and concentrated. Purified SIA-IgG Fabs were aliquoted until use.
3. C_{H}1-BSA: C_{H}1 domain conjugated with BSA as carrier (wherein N-glycosylated sialic acid modification at Asn162 site of the C_{H}1 domain).

Automatic solid-phase peptide synthesis was performed using Fmoc Rink Amide MBHA resin. The α N-Fmoc protected amino acid (from Gill Biochem) was used for peptide solid-phase synthesis, CS Biopeptide Synthesizer (CX136XT) was used for automatic peptide synthesis. Peptides were synthesized using DMF as a solvent and dissociated in piperidine/DMF (20/80, v/v) for 5 minutes (×2), followed by coupling with excess amino acids (4 equivalents) and HATU/HOBt (1:1, 4 equivalents) for 20 minutes. The coupling cycle of amino acids was performed based on the C_{H}1 amino acid sequence. After solid-phase synthesis, the resin loaded with the peptide was transferred to a peptide synthesis tube containing DCM for N-terminal modification (162 site), which involves cyclic reaction using different glycosyltransferases and corresponding glycosides (reaction temperature at 37° C.). After cycles, the glycopeptide can be obtained, and then the glycopeptide was eluted from the resin. BSA was then coupled onto the glycopeptide.

### Example 10. Immunization of Mice

1. SPF grade female BALB/c mice were randomly divided into 6 groups (5 mice/group):
Group 1: SIA-IgG;
Group 2: SIA-IgG Fab;
Group 3: C_{H}1-BSA;
Group 4: unimmunized group (control);
Group 5: SIA-IgG digested by sialidase (indicating that the sialic acid modification is removed from Asn162 site)
Group 6: SIA-IgG Fab digested by sialidase (indicating that the sialic acid modification is removed from Asn162 site).
2. SIA-IgG, SIA-IgG Fab, C_{H}1-BSA, SIA-IgG digested by sialidase, SIA-IgG Fab digested by sialidase were mix separately with Freund's complete adjuvant so as to prepare immunogenic compositions.
3. Preparation of polyclonal antibody:

Mice were immunized via foot pads with the immunogenic compositions of different groups (at 50 μg/mL protein). Unimmunized mice were used as controls and injected with equal volumes of PBS mixed with adjuvant. 2 weeks after the first immunization, the mice were subjected to a second immunization in the same manner. Two weeks later, blood was collected through the suborbital vein, and serum was separated and stored at −80° C. until use.
4. Preparation of monoclonal antibody:
1) Immunization of mice: Purified SIA-IgG (or SIA-IgG Fab or C_{H}1-BSA) was used to immunize mice with complete Freund's adjuvant (6-month-old mice, 100 μg/mouse). After three weeks, booster was performed, and SIA-IgG was used as antigen to dynamically analyze serum antibody titers. When antibody titer was greater than 1:10000, the mice were euthanized.
2) Cell fusion:

SP2/O myeloma cells in logarithmic growth phase were centrifuge at 1000 r/min for 5 minutes, the supernatant was removed, the cells were suspended in incomplete culture medium. The required number of cells were washed twice with medium. The myeloma cells and spleen cells were mixed at a ratio of 1:10 or 1:5, washed once with incomplete culture medium in a 50 ml plastic centrifuge tube, and centrifuged at 1200 r/min for 8 minutes. The supernatant was removed, any remaining liquid was removed to avoid affecting the concentration of PEG. Fusion was performed at room temperature, 1 ml of preheated 45% polyethylene glycol (PEG, Merek, relative molecular weight 4000, in 5% DMSO) was added within 30 seconds, and incubated for 90 seconds. The preheated incomplete culture medium was added to terminate PEG action. The cells were centrifuged at 800 r/min for 6 minutes, the supernatant was removed, and the cells were gently suspended with about 6 ml of RPMI1640 (comprising 20% calf serum). The fused cell suspension was added to 96 well culture plate containing feeder cells, at a concentration of 100 v 1/well, and incubated in a 37° C., 5% CO₂ incubator.
3) Hybridoma cell screening: Selective culture was performed using HAT medium. SP2/0 and splenic cells that were not fused were unable to survive in HAT medium, thus obtaining fused hybridoma.
4) Screening of positive clones: hybridoma cells were diluted in a 96 well culture plate to achieve only one cell per well. After proliferation, the supernatant was detected to identify the presence of specific antibodies. SIA-IgG was used as antigen and different culture supernatants were used as antibodies to be tested. The screened positive hybridomas were cultivated for further negative screening. SIA-IgG treated with neuraminidase was used as the antigen (for negative screening), and different culture supernatants were used as the antibodies to be tested. Positive hybridomas were collected. Fab of SIA-IgG or C_{H}1 with modification at Asn 162 site was used as the antigen, the culture supernatants after negative screening were used as the antibodies to be tested. The screened positive hybridoma was obtained for subcloning.
5) identification:
ELISA:

Fab of SIA-IgG or C_{H}1 with modification at Asn 162 site was used as the antigen, and the culture supernatant was used as the antibody to be tested to identify positive hybridoma;
Western blot:

Purified SIA-IgG, SIA-IgG treated with neuraminidase, Fab of SIA-IgG, and Fc were used as antigens. Under reducing condition (DTT treatment), the proteins were separated on SDS-PAGE and transferred onto NC membrane. After washing the membrane with PBS, the nitrocellulose membrane was blocked for 1 hour, and the nitrocellulose membrane was incubated with different clone supernatants at room temperature for 1 hour or 4° C. overnight. After washing the membrane with PBS, the membrane was incubated with anti-mouse IgG-HRP for 1 hour, the results were detected by chemiluminescence. The screened antibodies for SIA-IgG and SIA-IgG Fab shall be positive; for SIA-IgG treated with neuraminidase and Fc, the antibody should be negative.
Flow cytometry or cellular immunofluorescence:

RP215 was used as a positive control, epithelial derived tumor stem cells were used as test cells, and fibroblast cell lines were used as negative cells. The culture supernatant was incubated with the aforementioned cells, and mouse IgG-FITC was incubated with the cells for 30 minutes. The cells were loaded for flow cytometry or fluorescence microscopy detection. For tumor stem cells of epithelial origin, the screened antibodies should be positive, and for fibroblast lines, they should be negative.

### Example 11. Identification of Specific Antibodies

1. Antigen used for identification: C_{H}1 domain (wherein N-glycosylated sialic acid modification at Asn162 site of the C_{H}1 domain).
2. Indirect ELISA detection:

The 96-well plate was coated with 50 μl/well C_{H}1 domain and incubated overnight at 4° C. (1 mg/ml, diluted with 1 mM PBS PH7.2). The plate was rinsed for three times with PBS, and then blocked with 1% BSA for 2 hours. The coating reagent was removed and then gradient dilution of mice serum from 6 groups were added into the plate (starting from 1:200, diluted with 1 mM PBS pH 7.2); and the plate was incubated at 37° C. for 1 hour. The plate was rinsed for three times with PBS. Goat-anti-human IgG-HRP (1:5000, diluted with 1 mM pH 7.2 PBS containing 0.25% BSA) was added at 50 μl/well, and incubated at 37° C. for 30 minutes. The plate was rinsed for three times with PBS. 100 μl of TMB substrate solution was added and incubated at room temperature in dark for 15 minutes. 1 drop of 10% sulfuric acid/well was added to terminate the reaction. OD450 value was measured by using ELISA reader.
3. Results:

Mice were immunized twice with SIA-IgG, SIA-IgG Fab, SIA-IgG digested by sialidase, SIA-IgG Fab digested by sialidase, C_{H}1-BSA or PBS (as negative controls) respectively. The C_{H}1 domain was used as antigen for detecting the average titers of antibodies in the serum of the six groups.

It was found that, SIA-IgG (Group 1), SIA-IgG Fab (Group 2) and C_{H}1-BSA (Group 3) generated high titers of antibodies. However, groups 5 and 6 produced very low levels of antibodies (FIG. 8). This indicates that sialidase treatment resulted in the cleavage of N-glycosylated sialic acid at the 162 site in the C_{H}1 domain, leading to the loss of the epitope recognized by RP215 IgG (i.e. N-sialic acid at the 162 site). In addition, this embodiment also demonstrates that SIA-IgG SIA-IgG Fab and C_{H}1-BSA can both serve as effective immunogens to stimulate animal immune responses and produce antibodies. In short, SIA-IgG, SIA-IgG Fab, and C_{H}1-BSA can serve as immunogens and can be used to prepare immune complexes or vaccines, as well as to produce antibodies.

### Example 12. Repeatability of the Method

For SIA-IgG, SIA-IgG Fab and C_{H}1-BSA, the method of Example 11 was repeated to prepare antibodies. The method can yield higher titers of antibodies (data not shown). This indicates that SIA-IgG, SIA-IgG Fab and C_{H}1-BSA as immunogen show reproducibility in manufacture of antibodies.

Without relying on the amino acid sequences of the screened antibodies, SIA-IgG, SIA-IgG Fab and C_{H}1-BSA as immunogen make it possible to generate antibodies specific for SIA-IgG, SIA-IgG Fab or C_{H}1-BSA by well-known immune procedures, screening procedures, and titer-testing methods in the art.

### Example 13. Comparison of Antigen Preparation Method

The antigen preparation method disclosed herein is significantly better than the method of naturally isolating RP215. In order to isolate RP215, it is necessary to collect the cell lysate of ovarian cancer cell lines (i.e. a mixture of tumor proteins). Selecting specific monoclonal antibodies from hybridoma requires only purified recombinant SIA-IgG or glycosylated C_{H}1 as antigen. Compared with the preparation method of isolating RP215, the method herein has the following advantages:
(1) The antigen molecules and epitopes are clear, so the positivity rate of candidate hybridomas is greatly increased;
(2) The screening time has been significantly shortened;
(3) High affinity monoclonal antibodies targeting sialic acid at position 162 in C_{H}1 domain can be easily screened.

TABLE 1
Comparison of preparation method with RP215 Separation

| | | Monoclonal | | Obtain high |
|---|---|---|---|---|
| | | antibody | | affinity |
| | Antigen | positivity | | monoclonal |
| | purity | rate | duration | antibodies |
| RP215 | Mixture of | Low | Long | Hard |
| Separation | tumor | | | |
| | proteins | | | |
| | Low | | | |
| The antigen | SIA-IgG or | High | Short | Easy |
| preparation | glycosylated | | | |
| method | C_{H}1 domain | | | |
| disclosed | High | | | |
| herein | | | | |

### Example 14

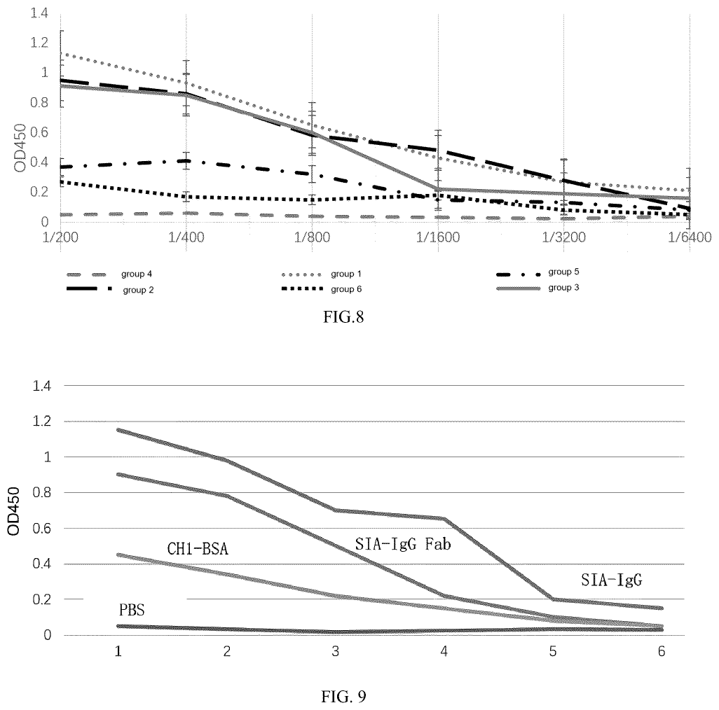

Three different immunogens (isolated SIA-IgG, SIA-IgG Fab and C_{H}1-BSA) were compared. It was found that SIA-IgG as an immunogen was better, followed by SIA-IgG Fab. C_{H}1-BSA was enough to generate specific antibodies, the antibody titer was relatively lower (FIG. 9) than that of SIA-IgG or SIA-IgG Fab.

### Example 15. Immunogenic Vaccines/Peptides for Human/Animal-Use

### Method:

1) Recombinant SIA-IgG was prepared in CHO or 293T; or C_{H}1 domain (wherein N-glycosylated sialic acid modification at Asn162 site of the C_{H}1 domain) was synthesized.
2) Sterilization treatment and virus inactivation.
3) 2-10 mg/kg SIA-IgG or glycosylated C_{H}1 domain was mixed with nanoadjuvant, and then subjected to quality check to ensure that the vaccine meets safety and efficacy standards.
4) The prepared vaccine was packaged and stored.

While the principle and implementation of the present invention are described in the above specific embodiments, those embodiments are only provided to facilitate understanding the core idea of the present invention. It should be noted that, for those of ordinary skill in the art, any obvious modifications, equivalent replacements, or other improvements made without departing from the inventive concept should be included in the scope of protection of the present invention.

1. An isolated immunogenic peptide, wherein the immunogenic peptide comprises the amino acid sequence as shown in SEQ ID NO: 1 with a N-glycosylated sialic acid modification at Asn162 site.

2. The isolated immunogenic peptide according to claim 1, wherein the Asn162 site represents site 162 in constant region of RP215-IgG heavy chain according to Kabat numbering criteria, the Asn162 site corresponds to the 13^{th} residue according to natural order in SEQ ID NO: 1.

3. The isolated immunogenic peptide according to claim 1, wherein the immunogenic peptide is a C_{H}1 domain of RP215-IgG with a N-glycosylated sialic acid modification at Asn162 site.

4. The isolated immunogenic peptide according to claim 1, wherein the immunogenic peptide is a Fab fragment of RP215-IgG with a N-glycosylated sialic acid modification at Asn162 site.

5. The isolated immunogenic peptide according to claim 1, wherein the immunogenic peptide is an RP215-IgG with a N-glycosylated sialic acid modification at Asn162 site.

6. An immunogenic conjugate, comprising:
the isolated immunogenic peptide according to claim 1; and
a carrier, wherein the carrier is selected from the group consisting of keyhole limpet hemocyanin (KLH), bovine serum albumin (BSA), OVA, maltose binding protein (MBP), flagellin, thyroglobulin, polymers of D- and/or L-amino acids, and tetanus toxoid; wherein
the carrier is conjugated to the isolated immunogenic peptide at an N-terminus or a C-terminus.

7. The immunogenic conjugate according to claim 6, wherein the carrier is BSA; and wherein the BSA is conjugated to the isolated immunogenic peptide at an N-terminus or a C-terminus.

8. An immunogenic composition, comprising:
an effective amount of the isolated immunogenic peptide according to claim 1 and a pharmaceutically acceptable excipient.

9. A method for inducing an immune response comprising:
administering to a non-human animal or a human an effective amount of the isolated immunogenic peptide according to claim 1.

10. A method for producing an antibody comprising:
administering to a non-human or a human an effective amount of the isolated immunogenic peptide according to claim 1.

11. The method of claim 10, wherein:
the antibody is a monoclonal antibody or polyclonal antibody.

12. An antibody, which is obtained by the method according to claim 10.

13. The antibody according to claim 12, wherein the antibody is specific for the C_{H}1 domain of RP215-IgG with a N-glycosylated sialic acid modification at Asn162 site.

14. An immunogenic composition, comprising:
an effective amount of the immunogenic conjugate according to claim 6 and a pharmaceutically acceptable excipient.

15. A method for inducing an immune response comprising:
administering to a non-human animal or a human an effective amount of the immunogenic conjugate according to claim 6.

16. A method for inducing an immune response comprising:
administering to a non-human animal or a human an effective amount of the immunogenic composition according to claim 8.

17. A method for producing an antibody comprising:
administering to a non-human or a human an effective amount of the immunogenic conjugate according to claim 6.

18. A method for producing an antibody comprising:
administering to a non-human or a human an effective amount of the immunogenic composition according to claim 8.

19. A method for producing an antibody comprising:
administering to a non-human or a human an effective amount of the immunogenic conjugate according to claim 6.

20. A method for producing an antibody comprising:
administering to a non-human or a human an effective amount of the immunogenic composition according to claim 8.
